(19) United States
(12) Patent Application Publication
(10) Pub. No.: US 2024/0379222 A1
(43) Pub. Date: Nov. 14, 2024

(54) VIRTUAL CARE MODULE

(71) Applicant: Aceso Interactive, Inc., Wilbraham, MA (US)

(72) Inventors: Geoff FIEDLER, Falmouth, MA (US); Todd BABINEAU, Wilbraham, MA (US); Dennis MATHUR, Massapequa, NY (US)

(73) Assignee: Aceso Interactive, Inc., Wilbraham, MA (US)

(21) Appl. No.: 18/658,361

(22) Filed: May 8, 2024

Related U.S. Application Data
(60) Provisional application No. 63/465,484, filed on May 10, 2023.

(51) Int. Cl. G16H 40/67; G06F 21/31; G06F 21/62; G16H 10/60; G16H 80/00

(52) U.S. Cl. CPC G16H 40/67; G06F 21/31; G06F 21/6245; G16H 10/60; G16H 80/00

(57) ABSTRACT

A method for facilitating video conferencing is provided. The method includes creating communication interface channels for receiving patient-related data from the transmission platform. The method includes receiving, through the communication interface channel, a message containing patient-related data from the transmission platform. The method includes authenticating the message. The method includes parsing the patient-related data contained in the authenticated message. The parsing includes converting the patient-related data into one or more data objects having a format compatible with the system. The method includes transmitting the standardized patient-related data to one or more in-room devices. The method includes configuring a connection between the in-room device and the transmission platform.

### CROSS-REFERENCE TO RELATED APPLICATION

The present application claims priority to Provisional Patent Application Ser. No. 63/465,484 filed on May 10, 2023, the entire disclosure of which is incorporated herein by reference.

### FIELD OF THE INVENTION

The subject matter disclosed herein generally relates to a virtual care module tailored for facilities in any number of industries. The disclosure focuses on the healthcare industry, describing systems, devices and methods for utilizing external video communications platforms within a protected healthcare hardware and software setting. It will be understood that the invention is not limited to applications in the healthcare industry but can also be applied to other industries as well including industries where tailored content is provided to specialized audiences, allowing for high-level interaction between those audiences and the system to advance institutional and other objectives.

### BACKGROUND OF THE INVENTION

As of 2022, there were over 6,000 registered hospitals, over 900,000 staffed beds, and over 33,000,000 patient admissions in the United States alone. (Source: American Hospital Association 2022 Fast Facts that can be found at https://www.aha.org/statistics/fast-facts-us-hospitals). Connecting virtually with a hospital inpatient has become increasingly important as healthcare professionals and family and friends of inpatients work and communicate virtually due to distance challenges, healthcare risks, efficiencies, personal preferences, and other reasons.

While many applications for video conferencing platforms exist today (FaceTime, Zoom, Skype, Google Meet, Microsoft Teams, GoToMeeting, Webex, FreeConference, etc.) and are utilized by professionals and the members of the general public, conducting such video conferences in a hospital setting presents a host of challenges.

For example, accessing the traditional video conferencing platforms using hospital provided tablets presents challenges because these tablets are difficult to tether to a bed, hold steady, and keep charged for use. Additionally, patients often run into issues utilizing traditional video conferencing platforms from their personal devices due to limitations caused by their health.

Traditional video conferencing platforms pose further challenges on clinicians because initiating calls to patients using these platforms requires that the clinician know the user-id/phone number to call. Additionally, traditional video conferencing platforms do not typically have integration with EHR to track the patient's location.

Further, some traditional video conferencing platforms require separate devices and monitors be mounted, powered, and networked into patient rooms, presenting further challenges for identifying which the correct device for routing a video call.

Currently, because of the above-described barriers for using traditional video conferencing platforms in a hospital setting, these platforms are typically utilized in a hospital setting by wheeling in mobile cart with a camera configuration. However, the traditional “cart” and camera configurations do not allow patients sufficient controls for toggling their camera and audio setting such that the patient is in control of the video feed originating from their hospital room. Additionally, using the traditional “cart” and camera configurations requires constant cleaning and disinfecting and increases the risk spreading infectious disease.

### SUMMARY OF THE INVENTION

There is therefore a need for a virtual care platform that utilizes commercial and other transmission platforms to, for example (in a healthcare setting), minimize potential exposure time to infectious diseases to reduce chance of spread; leverage resources in both inpatient and outpatient facilities to maximize total care touchpoints; allow providers to continue practicing while in isolation; alleviate provider burn-out by extending flexibility and care management remotely; eliminate the need to maintain and disinfect a wheel-in cart to providers can spend more time on treatment and communication; integrate with the in-room patient television providing a seamless all-in-one solution for delivering care without disrupting the patient experience; provide enhanced privacy for patients; and provide a modular solution that can be standalone or bundled.

Certain embodiments solve these problems with a significant advancement in integrating video communications platforms (including widely-used commercial platforms) into the controlled and secure hardware and software setting of an institution. Specifically, as shown and described herein, some embodiments provide a communication system for facilitating video conferencing that has one or more servers communicatively coupled to a transmission platform and one or more in-room devices, where the one or more servers parse data received from the transmission platform to allow video signals to be sent and received through the in-room devices. Overall, certain embodiments are therefore a significant advancement in the field of institution-based computer technology allowing users to achieve key outcomes including by allowing healthcare providers, patients, and loved ones of patients to communicate by utilizing widely used commercial and other video transmission platforms.

The above and other various aspects and embodiments are described below with reference to the accompanying drawings.

### BRIEF DESCRIPTION OF THE DRAWINGS

The accompanying drawings, which are incorporated herein and form part of the disclosure, help illustrate various embodiments of the present invention and, together with the description, further serve to describe the invention to enable a person skilled in the pertinent art to make and use the embodiments disclosed herein.

In the drawings, like reference numbers indicate identical or functionally similar elements.

FIG. 1 illustrates an interactive communications and control system within a healthcare institution as shown and described herein.

FIG. 2 illustrates another aspect of an interactive communications and control system within a healthcare institution, as shown and described herein.

FIG. 3 illustrates another aspect of an interactive communications and control system within a healthcare institution, as shown and described herein.

FIG. 4A illustrates another aspect of an interactive communications and control system within a healthcare institution, and devices utilized in that system, as shown and described herein.

FIG. 4B illustrates another aspect of devices utilized by an interactive communications and control system within a healthcare institution, as shown and described herein.

FIG. 4C illustrates another aspect of devices utilized by an interactive communications and control system within a healthcare institution, as shown and described herein.

FIG. 5A illustrates a user interface utilized by an interactive communications and control system within a healthcare institution, as shown and described herein.

FIG. 5B illustrates another aspect of a user interface utilized by an interactive communications and control system within a healthcare institution, as shown and described herein.

FIG. 5C illustrates another aspect of a user interface utilized by an interactive communications and control system within a healthcare institution, as shown and described herein.

FIG. 5D illustrates another aspect of a user interface utilized by an interactive communications and control system within a healthcare institution, as shown and described herein.

FIG. 5E illustrates another aspect of a user interface utilized by an interactive communications and control system within a healthcare institution, as shown and described herein.

FIG. 5F illustrates another aspect of a user interface utilized by an interactive communications and control system within a healthcare institution, as shown and described herein.

FIG. 5G illustrates another aspect of a user interface utilized by an interactive communications and control system within a healthcare institution, as shown and described herein.

FIG. 5H illustrates another aspect of a user interface utilized by an interactive communications and control system within a healthcare institution, as shown and described herein.

FIG. 5I illustrates another aspect of a user interface utilized by an interactive communications and control system within a healthcare institution, as shown and described herein.

FIG. 6 illustrates a method for the interactive sharing of information within a healthcare institution, as shown and described herein.

FIG. 7 illustrates another aspect of a method for the interactive sharing of information within a healthcare institution, as shown and described herein.

FIG. 8 illustrates another aspect of a method for the interactive sharing of information within a healthcare institution, as shown and described herein.

FIG. 9 illustrates another aspect of a method for the interactive sharing of information within a healthcare institution, as shown and described herein.

FIG. 10 illustrates another aspect of a method for the interactive sharing of information within a healthcare institution, as shown and described herein.

FIG. 11 illustrates another aspect of a method for the interactive sharing of information within a healthcare institution, as shown and described herein.

FIG. 12 illustrates another aspect of a method for the interactive sharing of information within a healthcare institution, as shown and described herein.

FIG. 13 illustrates another aspect of a method for the interactive sharing of information within a healthcare institution, as shown and described herein.

FIG. 14 illustrates another aspect of a method for the interactive sharing of information within a healthcare institution, as shown and described herein.

FIG. 15 illustrates another aspect of a method for the interactive sharing of

information within a healthcare institution, as shown and described herein.

FIG. 16 illustrates another aspect of a method for the interactive sharing of information within a healthcare institution, as shown and described herein.

FIG. 17 illustrates a method for the interactive sharing of information within a healthcare institution, as shown and described herein.

FIG. 18 illustrates a system for the control of media and communications systems, as shown and described herein.

FIG. 19A illustrates another aspect of a system for the control of media and communications systems as shown and described herein.

FIG. 19B illustrates another aspect of a system for the control of media and communications systems as shown and described herein.

FIG. 20 illustrates another aspect of a system for the control of media and communications systems as shown and described herein.

FIG. 21 illustrates a method for the control of media and communications systems, as shown and described herein.

FIG. 22 illustrates another aspect of an interactive communications and control system within a healthcare institution, as shown and described herein.

FIG. 23 illustrates a method for facilitating video communications within a healthcare institution, as shown and described herein.

FIG. 24 illustrates a method for facilitating video communications within a healthcare institution, as shown and described herein.

FIGS. 25A-J illustrate various user interfaces that can be utilized in connection with the system as shown and described herein.

FIG. 26 illustrates an aspect of the integration server as shown and described herein.

### DETAILED DESCRIPTION OF THE INVENTION

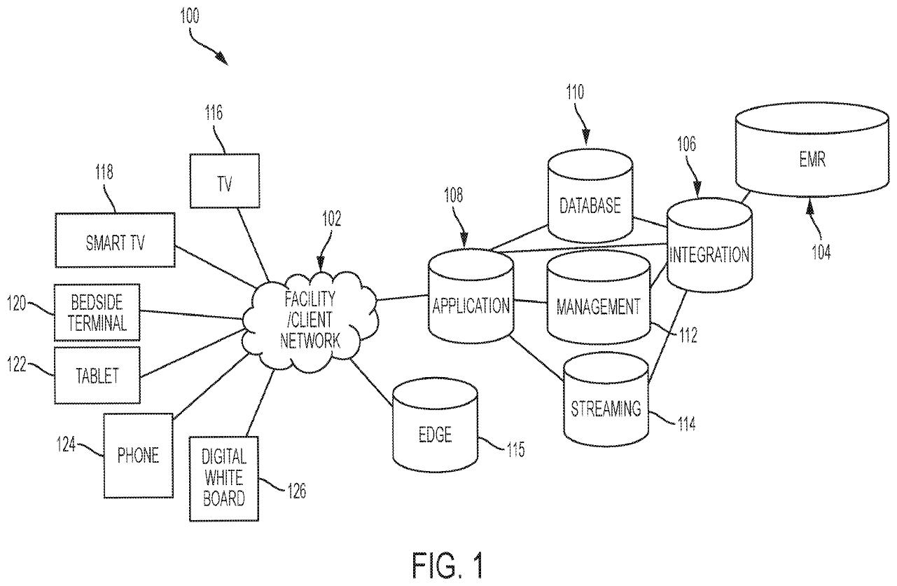

Referring now to FIG. 1, an embodiment of an electronic communications system 100 for the interactive sharing of information within a healthcare institution is shown.

The system 100 includes a number of hardware and software components designed to communicate across a local area network 102, wide area network, or other functional network to allow for the interactive sharing of information relating to a patient's care and stay at a healthcare institution.

In this embodiment, as will be described in more detail below, the components of the system 100 include an integration server 106, an application server 108, a database server 110, a management server 112, a streaming server 114, and one or more types of user devices including, but not limited to, a standard television 116, a smart television 118, a bedside terminal 120, a smart tablet 122, a smart phone 124, and a digital white board terminal 126.

These and other system 100 hardware and software components are configured to communicate with an electronic medical records (EMR) system 104, typically belonging to or otherwise operated by the healthcare or other institution where the system 100 is implemented. It will be understood that any number of other like components can be added to the system 100 to accomplish the objectives of the system 100 as described herein. It will also be understood that an edge server 115 can also be utilized when one or more of the other system 100 servers and other components are located off-site. The edge server 115 is configured to allow communication between such offsite system 100 components and onsite system 100 components in order to allow proper functioning of the system 100 as described herein.

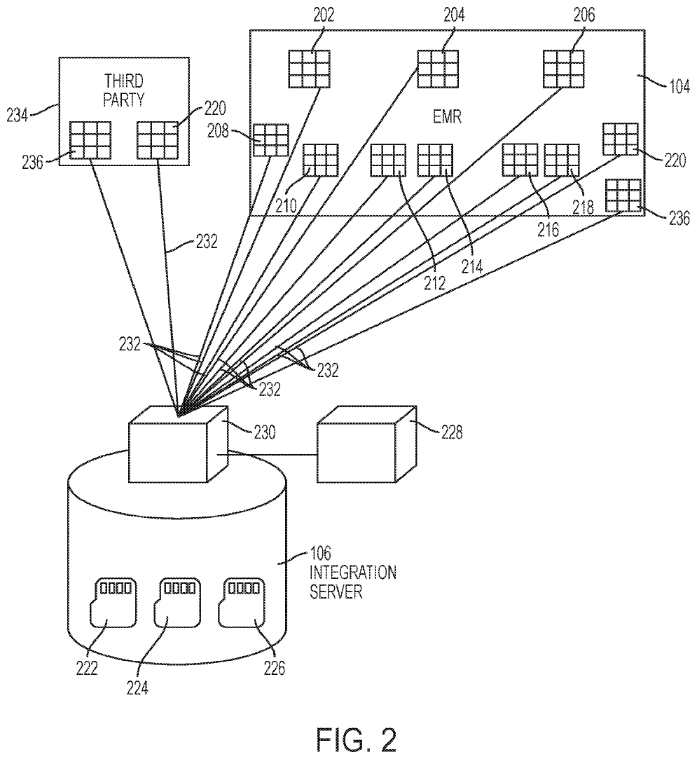

Referring now to FIG. 2, another aspect of the system 100 is shown. Here, each healthcare institution will have one or more EMR systems 104 that are institution-based full applications that store, have access to, and manage multiple data sets relating to each patient based on the workflows in a given healthcare institution or healthcare system. It will be understood that the EMR system 104 could utilize local servers, third party servers, or other components or databases configured to store, distribute and otherwise manage such data. It will be further understood that the EMR system 104 can reside on a single server or location, or reside on multiple servers or locations located within or outside of the facility in the vicinity of the system 100 or not.

The EMR system 104 contains information relating to a number of categories of patient and health information. This information is exchanged with system 100 through a variety of messages and transaction types relating to, among other things, patient ADT data (admissions, discharge, transfer and other patient-related data) 202, patient medication data 204, nursing assessment data 206, orders and results data 208, diet orders data 210, and patient education data 212 data.

In addition, the system 100 enhances the patient experience by providing patients with the ability to submit requests and feedback data 214, submit meal orders data 216, and submit HVAC control data (e.g., temperature control) 218 to the system 100 through user interfaces of each patient's device (e.g., smart television 118). This data, and other feedback data (e.g., relating to patient education feedback data 212) is channeled through the application server 108 and/or integration server 106, and exchanged with the EMR system 104, third party vendor applications, or other applications as appropriate.

The system 100 also enhances data received from the EMR system 104 when that data 220 might not be meaningful to a patient in the format in which it is stored on the EMR system 104. For example, patient diet orders data 210 received from the EMR system 104 as “NPO2” might be mapped in the EMR system 104 as: “Nil per os.” “Nil per os” is a Latin phrase meaning “nothing by mouth” restricting all oral food and fluids, and the system 100 is configured to present that data in plain English, or other languages, to the patient on one or more system devices.

Similarly, drug information 204 received from the EMR system 104 might have a variety of codes that the system 100 translates into plain language. For example, the EMR system 104 might provide a code: 23490647801, which the system will translate into “Warfarin” (the national NDC code for Warfarin is 23490647801). The system 100 is configured to query such information from third party vendors to obtain and display information in a patient-friendly format like “What is the drug used for”, “What are some adverse effects,” and display the corresponding information in plain language on one or more of the patient devices.

It will be understood that the above categories of information are provided as examples, and other categories of information could also be the subject of the operation of the system 100, devices and methods as described herein.

The system 100 also includes an integration server 106. In this embodiment, the integration server 106 is a virtual machine server configured to utilize one or more storage memory components 222 and utilize, interact with, or otherwise reside within the infrastructure of each given healthcare institution.

The storage memory components 222 can include one or more computer readable storage mediums 224 designed to store executable instructions such as computer code, and one or more other storage memory components 226 designed to store other data utilized during the operation of the system 100.

The integration server 106 is configured with a third party interface engine 230 that allows, in conjunction with an integration application program 228, for the receiving, sending and separation of a variety of messages and message types to and from the EMR system 104 and other system 100 servers and components using a variety of standards and protocols.

The integration application program 228 is also stored on the integration server 106 on one or more of its storage memory components 222, such as a computer readable storage medium 224, and is configured to communicate with the interface engine 230 to allow for the receiving, sending and separation of a variety of messages and message types to and from the EMR system 104 and other system 100 servers and components pursuant to the operation of the system 100 as described herein.

In the present embodiment, the interface engine 230 contains channels or pipes 232, which communicate with the EMR system 104, and contain operational instructions to access, parse and update information transacted with the EMR system 104 and other third party systems 234. Such information includes, but is not limited to, the ADT data (admissions, discharge and transfer) 202, medications data 204, nursing assessment data 206, orders and results data 208, diet orders data 210, education data 212, patient requests and feedback data 214, patient meal order 216 data, HVAC control data 218, patient friendly medication data 220, and other data 236.

The integration application program 228 is configured to communicate with the interface engine 230 and create and store parsed data sets corresponding to the accessed data sets, including parsed ADT data (admissions, discharge and transfer) 202, medications data 204, nursing assessment data 206, orders and results data 208, diet orders data 210, education data 212, patient requests and feedback data 214, meal ordering data 216, HVAC data 218, patient friendly medication data 220, and other corresponding data 236.

It will be further understood that the integration application program 228 operating on, or in communication with, integration server 106 is configured to act upon the parsed data identified above in a number of different ways depending upon the type and complexity of the messages and data in each data set. In some cases, the parsed data will be stored (on any of the storage memory components of the system 100). In other cases, the parsed data will be sent to the application server 108 for utilization in the system 100.

It will be further understood that the integration application program 228 operating on, or in communication with, integration server 106 is configured to receive data from the various system components (e.g., the application server 108) and in some circumstances communicate data, including updated information, to the EMR system 104.

The integration server 106 and integration application program 228 are also configured to communicate with the other servers, programs, and components of the system 100 across the network 102 in accordance with the operation of the system 100 as described herein.

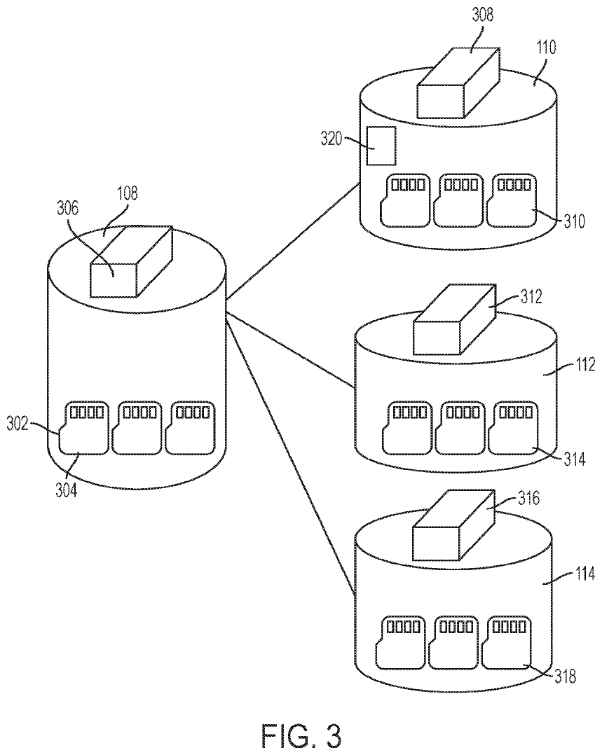

Referring now to FIG. 3 another aspect of the system 100 is shown. Here, the system 100 also includes an application server 108. In this embodiment, the application server 108 is a virtual machine server configured to utilize one or more storage memory components 302 and utilize, interact with, or otherwise reside within the infrastructure of each given healthcare institution.

The storage memory components 302 can include one or more computer readable storage mediums 304 designed to store executable instructions such as computer code, and one or more other storage memory components 302 designed to store other data utilized during the operation of the system 100.

The application server 108 is configured to store a system application program 306 on one or more of its storage memory components 302, such as a computer readable storage medium 304. The system application program 306 contains operational instructions to manage the operation of the system 100, including, but not limited to, sending, receiving and managing information amongst the various components of the system 100 to ensure proper operation of the system 100.

The system application program 306 is also configured to call, communicate with, or otherwise rely upon several other servers and programs within the system 100. For example, the system application program 306 is configured to call, communicate with, or otherwise rely upon integration server application program 228 residing on the integration server 106. This communication is important for, among other things, the sending, receiving and updating of data transacted with the facility EMR system 104, user interfaces and other components of the system 100.

The system application program 306 is also configured to call, communicate with, or otherwise rely upon a database server 110. In this embodiment, the database server 110 is a virtual machine server configured to store various system and patient data 320 including text and media (e.g., video) content, as well as certain operational computer code 308, on one or more storage memory components 310. The database server 110 is also configured to utilize, interact with, or otherwise reside within the infrastructure of each given healthcare institution.

The system application program 306 is also configured to call, communicate with, or otherwise rely upon a management server 112. In this embodiment, the management server 112 is a virtual machine server configured to store various system tools 315, as well as certain operational computer code 312, on one or more storage memory components 314. The management server 112 is also configured to utilize, interact with, or otherwise reside within the infrastructure of each given healthcare institution.

The system application program 306 is also configured to call, communicate with, or otherwise rely upon a streaming server 114. In this embodiment, the streaming server 114 is a virtual machine server configured to store video and other media content, tools to assist with the streaming of video content, as well as certain operational computer code 316, on one or more storage memory components 318. The streaming server 114 is also configured to utilize, interact with, or otherwise reside within the infrastructure of each given healthcare institution.

It will be further understood that the application program 306 operating on, or in communication with, application server 108 is configured to act upon the data it accesses and receives in accordance with the desired functioning of the system 100. In some cases the data will be stored, in other cases logical operations will be performed on the data, and in other cases the data will be relayed to other system 100 components for updating and execution.

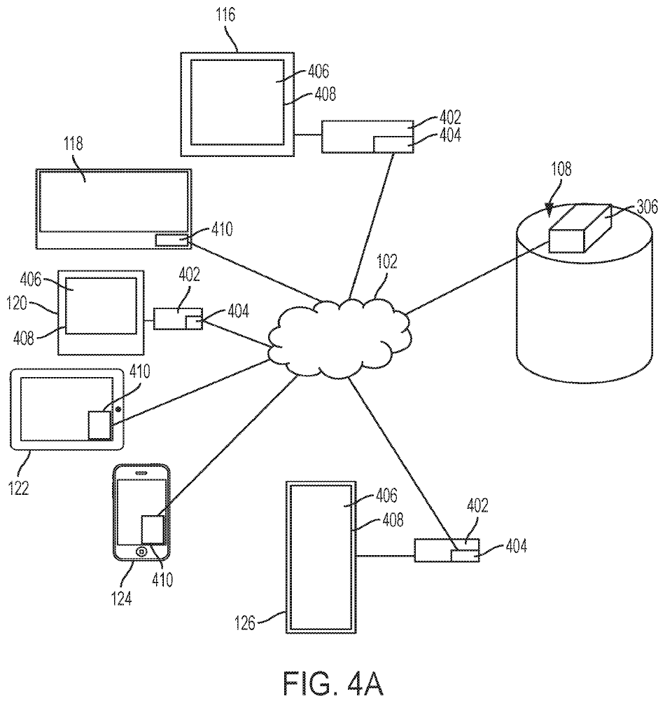

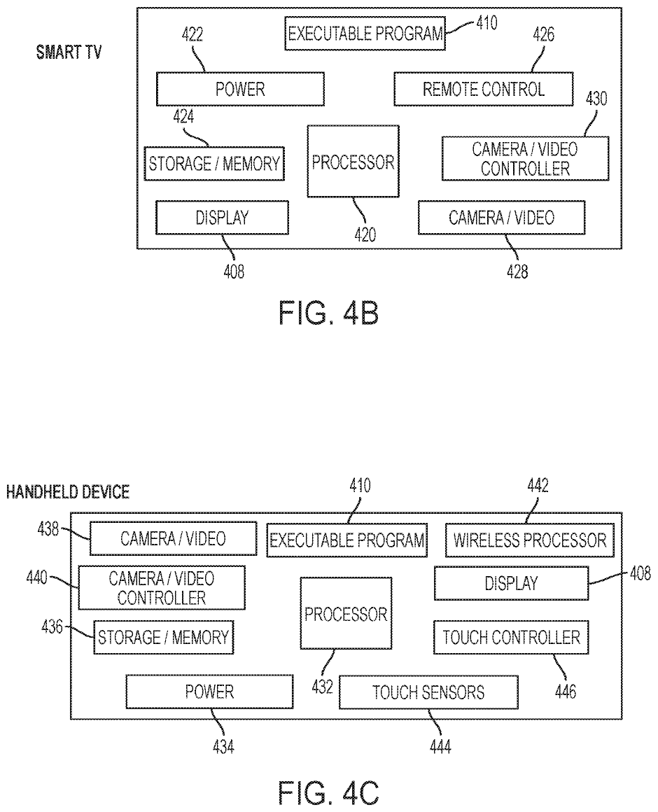

Referring now to FIG. 4A, another aspect of the system 100 is shown. Here, the system 100 includes various user, or endpoint, devices configured to allow end users (for example, patients, family members of patients, and healthcare institution personnel) to interact with and otherwise utilize the system 100.

The user devices can be in the form of a television 116, bedside terminal 120, and a dedicated digital white board terminal 126, each with an attendant television program box 402. In this embodiment, each television program box 402 is configured to store an executable program 404 that interfaces with the system application program 306 and allows the system application program 306 to communicate with the referenced devices and display the system's graphical user interface 406 on the display 408 of each device. It will be understood that with respect to the white board terminal 126, that terminal is configured, in one embodiment, to display key updated patient information on an ongoing basis in real time.

Similarly, the system 100 can also utilize a smart television 118, smart phones 124, smart tablets 122, or other intelligent devices that store an executable program 410 built into or otherwise stored upon each device, obviating the need for a program box such as program box 402 containing program 404.

In this embodiment, the executable programs 410 are configured to interface with the system application program 306 over the network 102 and allow the system application program 306 to communicate with the smart devices 118, 122 and 124 and display the system's graphical user interface 406 on the display 408 of each of the smart devices 168, 170 and 172.

It will be understood that the executable programs 404, 410 operating on or in conjunction with each user device are configured to also receive user input, such that each user of each device can input information actively (e.g., typing commands, selecting menu items, typing messages) or passively (e.g., tracked viewing habits), and provide (either sending or allowing access to) that information to the system application program 306 over the local area network 102, such that the system application program 306 can utilize that information in accordance with the operation of the system 100 described herein.

At this stage, it will be understood that the executable program 410 operating on, or in communication with, smart television 118 is configured to request information from the application server 108, including, but not limited to, information shown on the menu of each display (e.g., medication data 204, nursing assessment data 206, orders and results data 208, diet orders data 201, patient education data 212, patient requests and feedback data 214, meal ordering data 216, HVAC data 218, patient friendly medication data 220, and other data 236). It will be understood that the executable program 410 operating on, or in communication with, smart television 118 is configured to display this information on the user interface 408 located on the display 408 of the smart television 118, as the patient navigates through the various menu items.

It will be further understood that the executable program 410 operating on, or in communication with, smart television 118 is also configured to send active and passive information to the application server. Examples of active information include user-generated information (e.g., patient requests and feedback, meal ordering, HVAC requests) and passive user information (e.g., the time amount of a particular video a patient has watched, how many videos the patient has watched, how engaged is the patient with the various menu items in the system).

Referring now to FIG. 4B, further description of the components of a smart television 118 is shown. In this embodiment, the smart television 118 includes a number of components including, but not limited to, a display 408, executable program 410, processor 420, power supply (battery or hardwire) 422, storage memory component 424, remote control controller 426, camera (two-way video and audio capability) 428, and camera/video controller 430. It will be understood that these are examples of components utilized with the system 100, but it will be further understood that the smart television 118 can include any number of other components that can be utilized by the system 100 in the manners described herein and otherwise.

The executable program 410, tools, and APIs are stored on the device (e.g. smart television 118) allowing the executable program 410 to communicate with and control the device (including the device hardware components (e.g., to turn the device 118 on/off)). This executable program 410 for each device has the ability to recognize the device type (in this case a smart television 118) and apply the appropriate controls. The executable program 410 is configured to load/reload from the application server 108 for various activity including, but not limited to, patient admission and discharge, and other roadmaps specific to the healthcare industry, and to have constant communication with the rest of the system 100.

Referring now to FIG. 4C, further description of the components of a mobile phone 124 or tablet 122 devices is shown. In this embodiment, mobile phone 124 and/or tablet 122 includes a number of components including, but not limited to, a display 408, executable program 410, processor 432, power supply (battery or hardwire) 432, storage memory component 436, camera (two-way video and audio capability) 438, and camera/video controller 440, wireless processor 442, touch sensors 444 and touch controller 446. It will be understood that these are examples of components utilized with the system 100, but it will be further understood that the mobile phone 124 or tablet 122 devices can include any number of other components that can be utilized by the system 100 in accordance with the manners described herein and otherwise.

It will be further understood that each of the system 100 components contains non-transient computer readable storage mediums, or other storage mediums, on which such components are capable of storing information including executable and non-executable computer code, related source code, course code, binary files, application program interfaces (APIs), and/or other executable code or instructions.

It will be further understood that communication between any or all of the system 100 components can be performed through operation of one or more application program interfaces (APIs) contained within the system 100 allowing communication between system 100 components over a local area network 102, or wide area network when accessing components or information residing outside of the facility.

It will be further understood that the server arrangements described herein are provided as examples, but the arrangement of servers could be made in a multitude of other ways, including through one or more additional virtual machine servers with storage memory components operating within a cloud-based server network, and/or other server arrangements that would allow the system 100 to operate in the manners described herein.

It will be understood that the system's 100 utilization of virtual servers to operate the system 100 as described herein, in combination with the integration program 228, application program 306, and other components and applications of the system 100, provides the system 100 with many significant enhancements.

For example, this configuration, as described herein, provides the system 100 with the enhanced ability to operate within a broad range of diverse host environments and datacenters. It also provides the system 100 with enhanced disaster recovery, uptime and scalability. It also reduces the institution's data center footprint and allows for server consolidation. It also provides for easier maintenance, including enhanced ability to install a virtual machine server from an existing copy/template, re-installing, backup and movement to a different server. It also allows for maintaining scaled down test systems, and applying security patches in compliance with the policies of each institution.

It also eliminates the need to supply hardware on site, and provides a specialized information system unique to the healthcare industry (eliminating the need to adapt applications built to serve datacenter infrastructure of other industries, such as the hotel industry). It also eliminates the need to draw from disparate hardware and software sources and third party proprietary equipment to operate the system 100. It also allows for easier management and integration of endpoints (e.g., devices such as tablets, cell phones, televisions, etc.).

Moreover, it also eliminates the need to use specific video streaming hardware and rely on third party video streaming vendors. It also and allows the system 100 to use newer technologies that support lower and variable bandwidth for display on different types of portable devices, which further allows for a greater number of simultaneous video streams and hence serve a larger number of rooms/beds. It also provides the flexibility to separate out components to the local facility per the requirements of each institution (for example, in a typical deployment, a video streaming server is placed at a local facility, which saves on video bandwidth over the wide area network, or the system could flex between streaming video from the datacenter and local facility based on availability). It will be understood that these are examples of enhancements that the current invention provides to the computer technology previously utilized for such applications, and that other enhancements also exist inherently or otherwise.

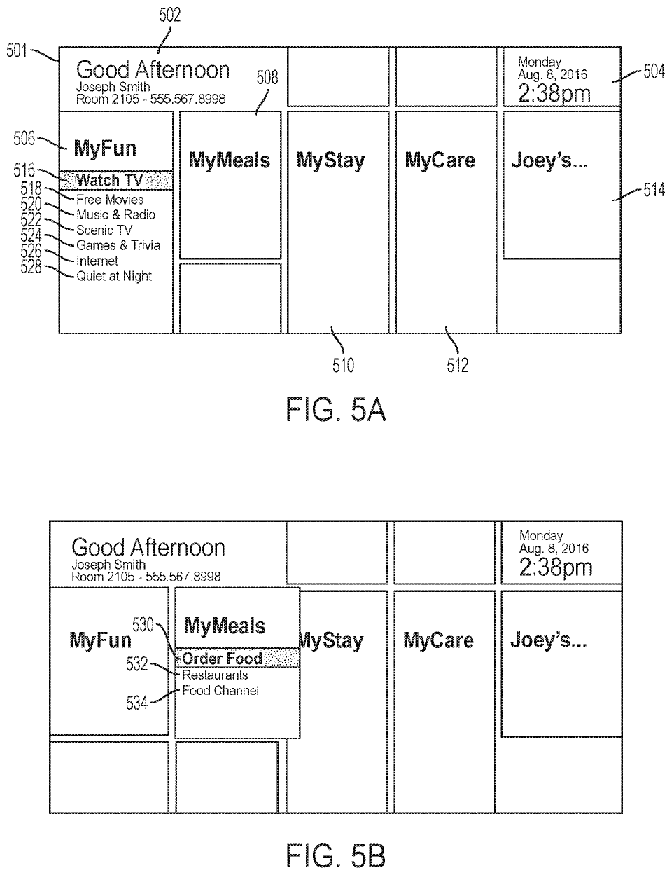

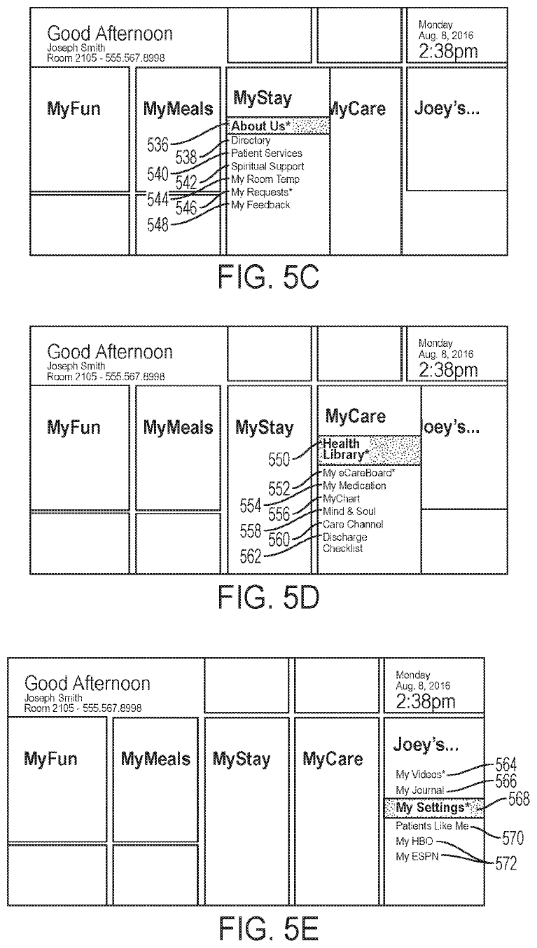

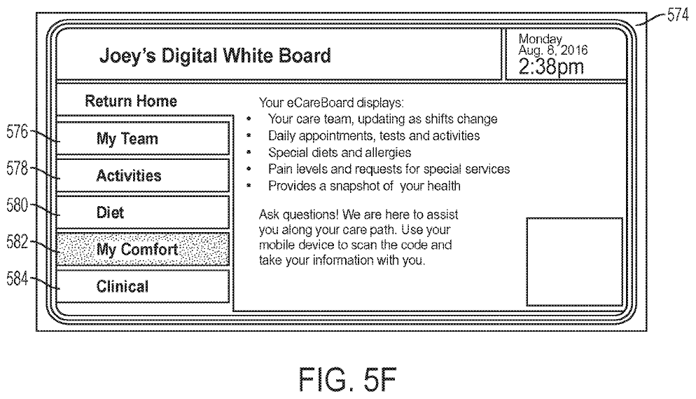

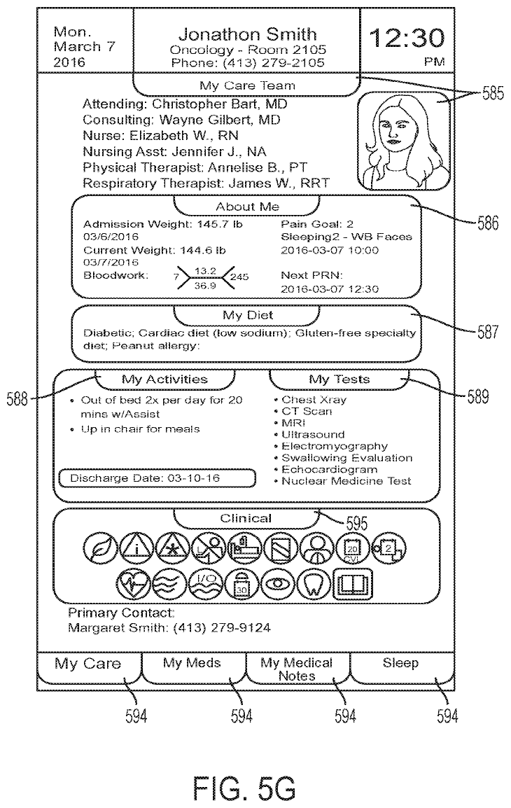

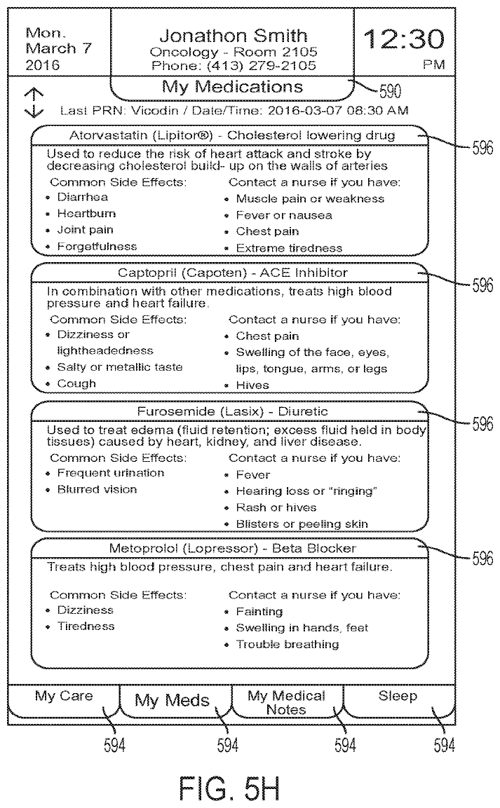

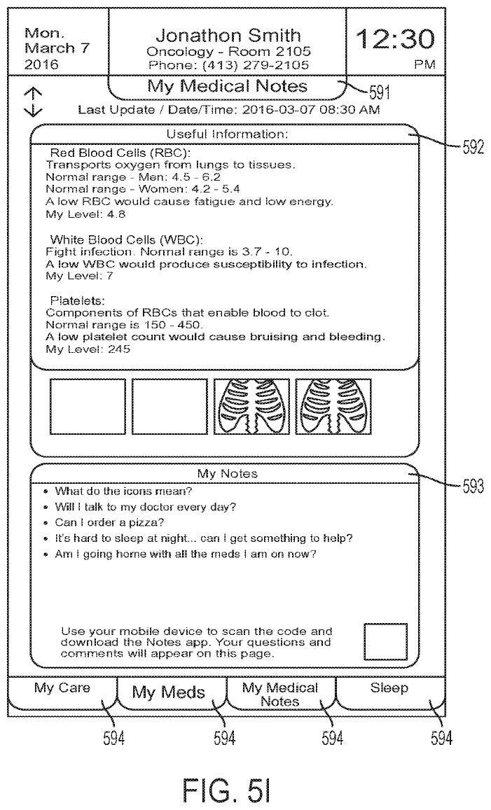

Referring now to FIG. 5A-5J, several embodiments of the user interface 406 displayed by the system application program 306 on each user device is shown. In these embodiments various menu items appear to a given user. In FIGS. 5A-F, these menu items and other offerings on the user interface 406 represent an entire feature set 501, providing the user with a multitude of offerings within the user interface 406.

It will be understood that one example of a feature set could be all of the elements and menu offerings shown and described in connection with FIGS. 5A-5F, and could also include many other features offered to a patient through the user interface 406 viewable on the display of the terminal or device. In the embodiments of FIGS. 5A-5F, the user can select actionable menu items in any manner allowed by the subject device, including, but not limited to, selection through remote control (such as through a television), selection through haptic contact engagement (such as through contact on the display of a smart phone or smart tablet), or selection through point-and-click (such as through a computer mouse and computer).

With respect to FIGS. 5G-5I, an embodiment of a graphical user interface 406 for a digital white board 126 is shown. It will be understood that this information is designed to display key patient information in real time, and it utilized by the patient and healthcare professionals, and has distinct functionality from the feature set displayed by the user interfaces set out in 5A-5F.

Referring now to FIG. 5A, an embodiment of a system user interface 406 is shown. Here, patient name and room information 502 is displayed, along with the day, date and time 504. General menu items relating to enjoyment 506, meals 508, stay 510, care 512, and personalized information 514 are displayed. The enjoyment menu item 506 can have subset menu items for the patient to choose from including general television 516, free movies 518, scenic television 522, games and trivia 524, internet 526, and sleep-related content 528. It will be understood that once selected, each actionable menu item will direct the patient to a new page allowing that user to further interact with the system 100 with respect to subject matter relating to enjoyment.

Referring now to FIG. 5B, another embodiment of a system user interface 406 is shown. Here, the meals menu item 508 can have subset menu items for the patient to choose from including ordering food based on their diet restrictions 530, identifying local restaurants 532, and viewing content from designated food channels 534. It will be understood that once selected, each actionable menu item will direct the patient to a new page allowing that user to further interact with the system 100 with respect to subject matter relating to meals.

Referring now to FIG. 5C, another embodiment of a system user interface 406 is shown. Here, the stay menu item 510 can have subset menu items for the patient to choose from including key institution information 536, institution directory 538, patient services 540, spiritual support 542, room temperature 544, special requests 546, and patient feedback 548. It will be understood that once selected, each actionable menu item will direct the patient to a new page allowing that user to further interact with the system 100 with respect to subject matter relating to the patient's stay.

Referring now to FIG. 5D, another embodiment of a system user interface 406 is shown. Here, the care menu item 512 can have subset menu items for the patient to choose from including health education library 550, digital whiteboard 552, medication 554, patient-specific chart 556, mind and soul 558, care channel (relaxing content) 560, and discharge checklist 562. It will be understood that once selected, each actionable menu item will direct the patient to a new page allowing that user to further interact with the system 100 with respect to subject matter relating to care.

Referring now to FIG. 5E, another embodiment of a system user interface 406 is shown. Here, the personalized information menu item 514 can have subset menu items for the patient to choose from including specifically assigned educational videos 564, patient journal 566, system settings 568, analogous patient cases 570, and tailored entertainment 572. It will be understood that once selected, each actionable menu item will direct the patient to a new page allowing that user to further interact with the system 100 with respect to subject matter relating to personalized patient information.

Referring now to FIG. 5F, another embodiment of a system user interface 406 is shown. Here, a virtual white board interface 574 is shown, being displayed within the navigable user interface 406. Here, the information displayed includes detailed information concerning a patient's care team 576, daily appointments, tests and activities 578, special diets and allergies 580, pain levels and requests for special services 582, and medications, test results, and other clinical information 584.

Referring now to FIG. 5G, an embodiment of a system user interface 406 for a digital white board 126 is shown. Here, the information displayed includes detailed information concerning a patient's care team 585, physiology 586, diet 587, activities and schedule 588, tests 589, and other clinical information 595. Subject matter tabs 594 for each set of data are provided on the display of the digital white board 126.

Referring now to FIG. 5H, another embodiment of a system user interface 406 for a digital white board 126 is shown. Here, the information displayed includes detailed information concerning a patient's medication 590, which could be a variety of medications falling under different fields 596. Subject matter tabs 594 for each set of data are provided on the display of the digital white board 126.

Referring now to FIG. 5I, another embodiment of a system user interface 406 for a digital white board 126 is shown. Here, the information displayed includes detailed information concerning key medical notes 591 for the patient, including a general category of useful clinical information 592, as well as other notes 593. Subject matter tabs 594 for each set of data are provided on the display of the digital white board 126.

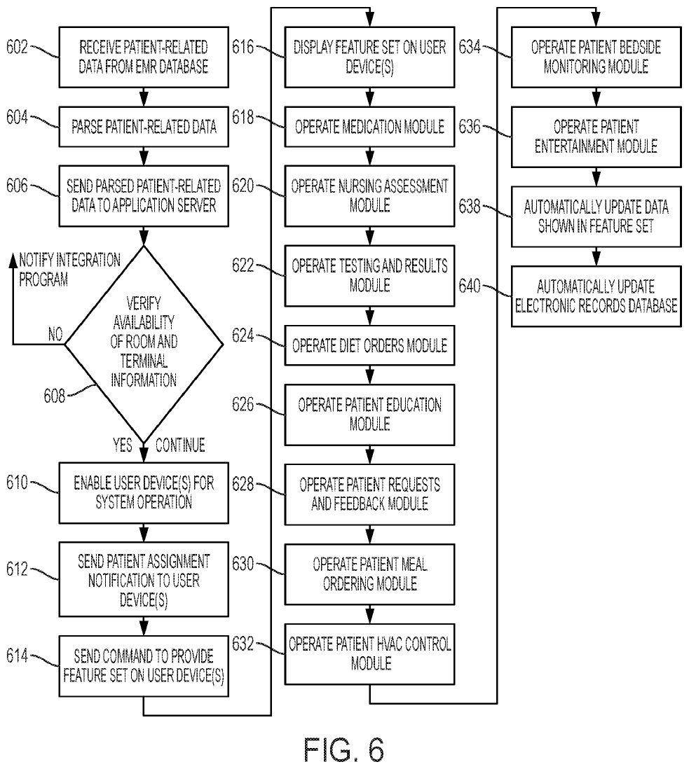

Referring now to FIG. 6, an embodiment of an interactive sharing of patient-related information method 600 is shown.

In step 602 of method 600, the integration application program 228 operating on, or in communication with, integration server 106 receives the ADT data 202, medications data 204, nursing assessment data 206, orders and results data 208, diet ordering data 210, education data 212, patient requests and feedback data 214, meal ordering data 216, HVAC (temperature control) data 218, patient friendly medication data 220, and other data 236 from the institution's EMR system 104.

In step 604 of method 600, the integration application program 228 operating on, or in communication with, integration server 106 parses the ADT data 202, medications data 204, nursing assessment data 206, orders and results data 208, diet ordering data 210, education data 212, patient requests and feedback data 214, meal ordering data 216, HVAC (temperature control) data 218, patient friendly medication data 220, and other data 236.

In step 606 of method 600, the integration application program 228 operating on, or in communication with, integration server 106 sends the parsed ADT data 202 to the application server 108. It will be understood that the ADT data 202 includes admission, discharge, transfer, and room information for each patient, as well as other patient data including, but not limited to, the patient's name, identifying information, demographics, physicians assigned to the patient, and other relevant information for that patient. The integration application program 228 passes all of this information to the application server 108 and the application server 108 stores that information as part of the patient's record.

In step 608 of method 600, the system application program 306 operating on, or in communication with, application server 108 verifies whether the room identified in the parsed ADT data 202 for a given patient is available. If such room is available, the method 600 continues. If not, the system application program 306 notifies the integration application program 228 and the method 600 ceases until the ADT data 202 is updated or otherwise corrected and the method 600 commences again beginning at step 602. It will be understood that the terminals 120 and other in-room devices are associated with certain rooms and beds in the institution during the initial configuration of the system 100. If for some reason a terminal 120 or device is not properly associated with a certain room and bed, or is replaced, the system application program 306 will provide a message of “Service Not Available” viewable on the user terminal or device, until the terminal or device is properly assigned to the appropriate room and bed.

In step 610 of method 600, if verified, the system application program 306 operating on, or in communication with, application server 108 enables the one or more devices and/or terminals in the assigned room to receive and otherwise display the patient-related information from the system 100. In this embodiment, the method 600 will utilize a single smart television 118 assigned to a certain room for a certain patient. However, it will be understood that multiple devices configured to be utilized by the system 100 and users of the system 100 could also be used as a terminal or device component of the system 100 in the manner described herein.

In step 612 of method 600, the system application program 306 operating on, or in communication with, application server 108 sends or pushes a notification to the smart television 118 that a certain patient is assigned to that device.

In step 614 of method 600, the system application program 306 operating on, or in communication with, application server 108 sends or pushes a command to the smart television 118 that the smart television 118 make the appropriate feature set 501 available on the smart television 118 for that patient.

In step 616 of method 600, the executable program 410 operating on, or in communication with, smart television 118 displays the feature set 501 on the user interface 408 located on the display 408 of the smart television 118. It will be understood that the feature set 501 in this embodiment includes, but is not limited to, the feature set 501 shown and described in FIG. 5, including all menu items, date and time information, and other information that the system application program 306 operating on, or in communication with, application server 108 sends to and receives from the smart television 118 to populate and otherwise link to the menu items on the user interface 406 as the user operates the system 100.

It will be understood that with respect to other smart devices, such as smart phones 124 and smart tablets 122, such devices are configured to receive a verification code from the system 100 to confirm the device belongs to that patient and is allowed access. It will be further understood that such access will be restricted to the patient room only, and would not be allowed once the device is removed from the room through a combination of local WiFi or geo-location mechanism, in which case the smart phones 124 and smart tablets 122, could contain general information and media but no patient-specific information.

At this stage, it will be understood that the smart television 118 is now connected to and otherwise operational within the system 100. It will be further understood that any number of different methods exist in connection with the operation of the system with respect to each feature of the feature set 501. For example, the system components can be configured to operate certain modules of each feature set 501.

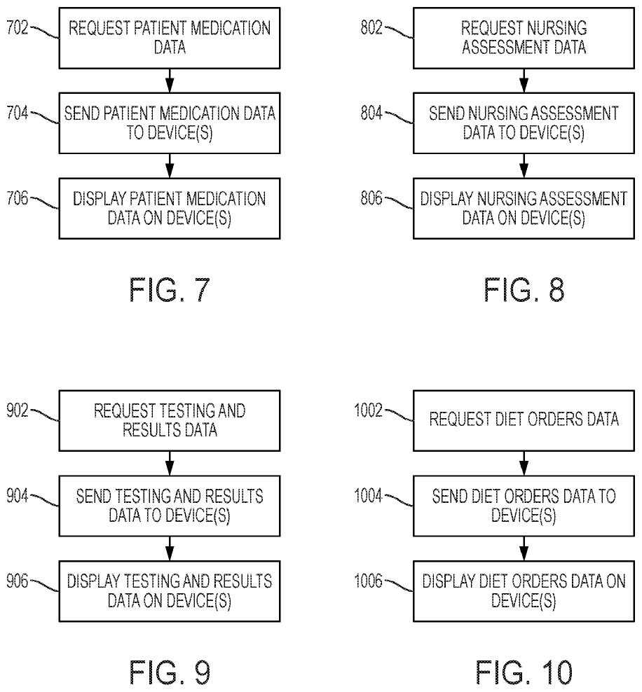

Accordingly, in step 618 of method 600, the system 100 operates a feature set based on medication data 204, more fully shown and described in reference to FIG. 7.

In step 620 of method 600, the system 100 operates a feature set based on nursing assessment data 206, more fully shown and described in reference to FIG. 8.

In step 622 of method 600, the system 100 operates a feature set based on orders and results data 208, more fully shown and described in reference to FIG. 9.

In step 624 of method 600, the system 100 operates a feature set based on diet orders data 210, more fully shown and described in reference to FIG. 10.

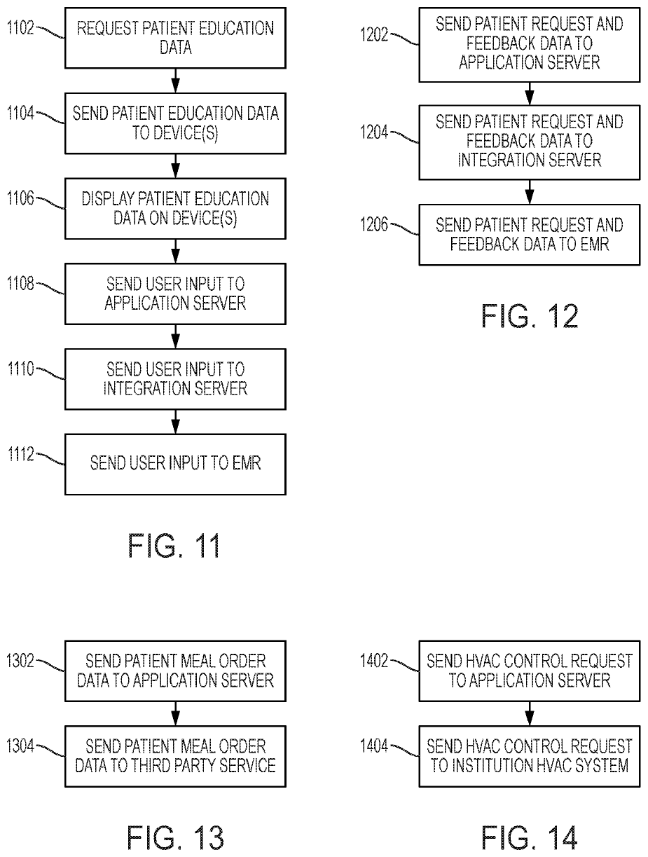

In step 626 of method 600, the system 100 operates a feature set based on patient education data 212, more fully shown and described in reference to FIG. 11.

In step 628 of method 600, the system 100 operates a feature set based on patient requests and feedback data 214, more fully shown and described in reference to FIG. 12.

In step 630 of method 600, the system 100 operates a feature set based on patient meal ordering data 216, more fully shown and described in reference to FIG. 13.

In step 632 of method 600, the system 100 operates a feature set based on patient HVAC control requests 218, more fully shown and described in reference to FIG. 14.

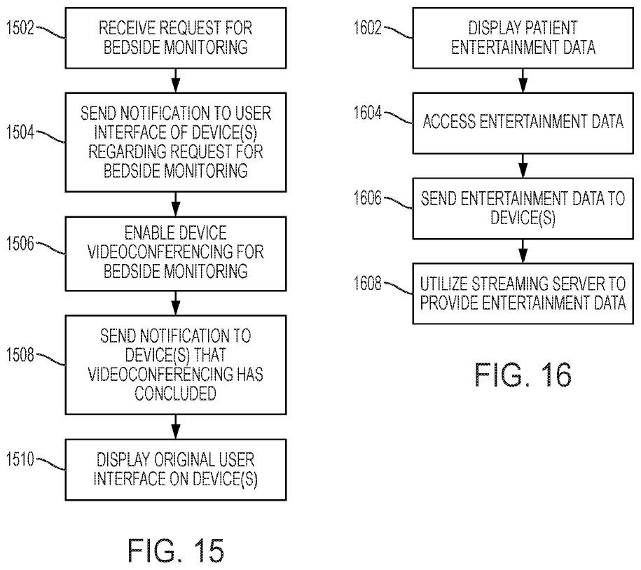

In step 634 of method 600, the system 100 operates a feature set based on patient bedside monitoring requests, more fully shown and described in reference to FIG. 15.

In step 636 of method 600, the system 100 operates a feature set based on patient entertainment, more fully shown and described in reference to FIG. 16.

In step 638 of method 600, the information contained in each feature set 501 described herein is continually updated as each of the feature set 501 menu items and other items are accessed. It will be understood that the updating of the underlying data occurs automatically based on communication between the various system components, including between the application server 108, integration server 106, and facility EMR system 104.

In step 640 of method 600, the information contained in the facility EMR system 104 is continually updated based on information received from the integration program 228 operating on, or in communication with, integration server 106 (based on information received from other system 100 components including the application server 108) based on certain activities by the patient.

It will be further understood that the modules described herein are provided as examples, and that other modules and features could also be understood by one of ordinary skill in the art to be within the scope of the invention as described herein.

Referring now to FIG. 7, steps for a medication data module of certain embodiments is shown and described in accordance with method 700. In step 702 of method 700, the executable program 410 operating on, or in communication with, smart television 118 requests patient medication data 204 from the application server 108 as a result of the user selection of the medication menu item 554.

In step 704 of method 700, the system application program 306 operating on, or in communication with, application server 108 sends the patient medication data 204 to the smart television 118. It will be understood that the integration program 228 operating on, or in communication with, integration server 106 parses the medication data 204 orders received from the EMR system 104 for key identifying information that is then used to query systems from other vendors to obtain, parse and store patient-friendly information. The integration program 228 operating on, or in communication with, integration server 106 sends this information to the application server 108. The application program 306 operating on, or in communication with, application server 108 sends the patient medication data 204 to the smart television 118 for display on the user interface 406 on the in-room device. Other activities such as immediate discontinuation of certain medications, updates to discontinuation dates in the future, and other key information items relating to patient medication data 204 are updated and/or modified in the system 100 accordingly.

In step 706 of method 700, the executable program 410 operating on, or in communication with, smart television 118 displays the medication data 204 on the display 408 of the smart television 118.

Referring now to FIG. 8, steps for a nursing assessment data module of some embodiments is shown and described in accordance with method 800. In step 802 of method 800, the executable program 410 operating on, or in communication with, smart television 118 requests nursing assessment data 206 from the application server 108 as a result of the user selection of the nursing assessment data from the care menu item 512.

In step 804 of method 800, the system application program 306 operating on, or in communication with, application server 108 sends the nursing assessment data 206 to the smart television 118. It will be understood that the nursing assessment data 206 includes, but is not limited to, nursing assessments and documentation including patient activity, goals, pain scale, and other data.

It will be further understood that the integration program 228 operating on, or in communication with, integration server 106 evaluates and acts upon the nursing assessment data 206 received from the EMR system 104. This includes assessment of the type of information received from the EMR system 104, updating the patient's clinical records within the system 100 appropriately, referencing patient friendly text as well as visual cues (fall risk, hearing aid, etc.) that is extensively mapped in the system 100 and database 110 design along with rules to handle updates for the different types of activity generated. For certain institutions, EMR systems 104, staff assignments and shifts might also be part of these records.

In step 806 of method 800, the executable program 410 operating on, or in communication with, smart television 118 displays the nursing assessment data 206 on the display 408 of the smart television 118. It will be understood that the nursing assessment data 206, as well as other patient-related data referenced herein, including in connection with FIGS. 5G, H and I, can also be displayed on the digital white board 126. In that configuration, the system application 306 operating on, or in communication with, application server 108 causes the information displayed on the digital white board 126 to be updated automatically in real time based on information changes at the EMR system level 104.

Referring now to FIG. 9, steps for an orders and results data module of certain embodiments is shown and described in accordance with method 900. In step 902 of method 900, the executable program 410 operating on, or in communication with, smart television 118 requests orders and results data 208 from the application server 108 as a consequence of the user selection of the orders and results data 208 from the care menu item 512.

In step 904 of method 690, the system application program 306 operating on, or in communication with, application server 108 sends the orders and results data 208 to the smart television 118. It will be understood that the orders and results data 208 includes, but is not limited to, data relating to orders for patients such as radiology (X-Rays, CT scans, MRIs), cardiology (ECG), and laboratory tests. It will be further understood that this information originates in the institution EMR system 104 and is parsed by the integration application program 228 and sent to and stored by the application server 108, such that the application program 306 operating on, or in communication with, application server 108 can send the data to the smart television 118 in accordance with the invention as described herein.

In step 906 of method 900, the executable program 410 operating on, or in communication with, smart television 118 displays the orders and results data 208 on the display 408 of the smart television 118.

Referring now to FIG. 10, steps for a diet orders data module of an embodiment is shown and described in accordance with method 1000. In step 1002 of method 1000, the executable program 410 operating on, or in communication with, smart television 118 requests the diet orders data 210 from the application server 108 as a result of the user selection of the diet orders data 210 from the meals menu item 508.

In step 1004 of method 1000, the system application program 306 operating on, or in communication with, application server 108 sends the diet orders data 210 to the smart television 118. It will be understood that the diet orders data 210 includes, but is not limited to, recommended or required diet order from healthcare personnel based on a patient's condition (e.g., “no eating for 12 hours before surgery,” “no sugar,” “fluids only”). It will be further understood that diet orders data 210 received from the EMR system 104 can have certain codes that are mapped to patient friendly information within the system 100. The application program 306 operating on, or in communication with, application server 108 looks up these codes and stores these codes against the patient's clinical information for display on the user interface 406 on the in-room device.

In step 1006 of method 1000, the executable program 410 operating on, or in communication with, smart television 118 displays the diet orders data 210 on the display 408 of the smart television 118.

Referring now to FIG. 11, steps for patient education feature of some embodiments is shown and described in accordance with method 1100. In step 1102 of method 1100, the executable program 410 operating on, or in communication with, smart television 118 requests patient education data 212 from the application server 108 as a result of the user selection of patient education data 212 from the personalized information menu item 514.

In step 1104 of method 1100, the system application program 306 operating on, or in communication with, application server 108 sends the requested patient education data 212 to the smart television 118. It will be understood that the data 212 is available to the system application program 306 because the integration program 228 operating on, or in communication with, integration server 106 continually receives relevant education data 212 from the EMR system 104, parses that data, and automatically sends that parsed data to the application server 108.

It will be further understood that the patient education feature of the system 100 is functionally bi-directional. For example, patient education data 212 (including videos, articles, and other information) can be initiated by institution professionals and staff at the EMR system 104 level. In putting the education data 212 together, institution professionals can draw from multiple sources including, but not limited to, physician orders, problem lists, medications, educational videos, and other sources.

It will be further understood that patient education data 212 content to be viewed (e.g., an educational video) is mapped in the system 100 along with questions on who is going to view the content and followed by questions to document patient comprehension. It will be further understood that the patient education data 212 can be provided in the form of educational video packets, or highly customized, prepared and vetted video and/or textual information relating to the specific condition, procedure, or other health-related item specifically relating to each patient, to be provided to each patient at the proper time.

In step 1106 of method 1100, the executable program 410 operating on, or in communication with, smart television 118 displays the patient education data 212 on the display 408 of the smart television 118. It will be understood that when a patient views the appropriate patient education data 212 content through the user interface 406, information relating to that viewing (e.g., if a video, whether viewing of a video was completed, and if not, how long the video was viewed) is passed back to the application server 108.

In step 1108 of method 1100, the executable program 410 operating on, or in communication with, smart television 118 sends the user input relating to the patient education data 212 to the application server 108.

In step 1110 of method 1100, the system application program 306 operating on, or in communication with, application server 108 sends the user input relating to the patient education data 212 to the integration server 106 where the proper message format is created in the interface engine 230.

In step 1112 of method 1100, the integration application program 228 operating on, or in communication with, integration server 106 sends the user input relating to the patient education data 212 to the EMR system 104 for documentation.

Referring now to FIG. 12, steps for a patient requests and feedback of certain embodiments is shown and described in accordance with method 1200. In step 1202 of method 1200, the executable program 410 operating on, or in communication with, smart television 118 sends patient requests and feedback data 214 to the application server 108 as a result of the user sending such data through the appropriate user menu item.

In step 1204 of method 1200, the system application program 306 operating on, or in communication with, application server 108 sends the patient requests and feedback data 214 to the integration server 106 where the proper message format is created in the interface engine 230.

It will be understood that the patient requests and feedback data 214 are messages that result from a patient selecting an item on the user interface 406 and are sent from there to the application server 108 which stores relevant information and sends that information to the integration server 106. It will be further understood that patient requests include, but are not limited to, any number of patient requests, such as wanting to speak to a pharmacist, wanting to speak with clergy, wanting a pillow, requesting bedside delivery of discharge medications, and the like. Requests such as for bedside delivery of discharge medications might be formatted as orders or results messages and sent back to the EMR system 104 for documentation and appropriate action. Similarly patient feedback, surveys, service recovery can be reported in a variety of formats to messaging systems and/or applications (like Vocera), or email, text message, and the like.

In step 1206 of method 1200, the integration application program 228 operating on, or in communication with, integration server 106 sends the patient requests and feedback data 214 to the EMR system 104 so that appropriate action can be taken.

Referring now to FIG. 13, steps for a patient meal ordering data module of some embodiments is shown and described in accordance with method 1300. In step 1302 of method 1300, the executable program 410 operating on, or in communication with, smart television 118 sends patient meal orders 216 to the application server 108 as a result of the user sending requesting such meal orders 216 through the user meals menu 508.

In step 1304 of method 1300, the system application program 306 operating on, or in communication with, application server 108 sends the patient meal orders 216 to a third party food service interface. It will be understood that such web-based food services provide dietary and nutrition services and systems to a given institution. The display and categorization of meals can be designed for each user interface 406 for each institution. In this embodiment, as the patient navigates the user interface 406, meals specific to the patient's diet are dynamically obtained through the user interface 406 and rules and restrictions are computed before the patient makes the final selection that is then passed back to the appropriate dietary and nutrition services and systems.

Referring now to FIG. 14, steps for a patient HVAC control module of certain embodiments is shown and described in accordance with method 1400. In step 1402 of method 1400, the executable program 410 operating on, or in communication with, smart television 118 sends patient HVAC control requests 218 to the application server 108 as a result of the user sending such HVAC control requests 218 (e.g., increase room temperature) through the stay menu item 510.

In step 1404 of method 1400, the system application program 306 operating on, or in communication with, application server 108 sends the patient HVAC control requests 218 data to the institution HVAC system. It will be understood that the integration server 106 communicates with the application server 108 when ADT data 202 messages are received to request room temperatures be set to default values.

Referring now to FIG. 15, steps for a patient bedside monitoring module of some embodiments is shown and described in accordance with method 1500. In step 1502 of method 1500, the system application program 306 operating on, or in communication with, application server 108 receives a request for bedside monitoring from a third party video conferencing system.

In step 1504 of method 1500, the system application program 306 operating on, or in communication with, application server 108 sends a notification to the user interface 406 of the smart television 118 to switch the smart television's 118 input to the appropriate source to receive the video conference. It will be understood that the user consent for this step is not required, and a device may be “hijacked” such that the healthcare professional can view the patient in a videoconferencing setting at any time the healthcare professional deems such a virtual visit is warranted. It will be further understood that additional components attached to the smart television 118 is configured to allow two-way audio and video communication between healthcare professional, patient, and patient guardians where applicable, through its various hardware and software features as shown and described herein.

In step 1506 of method 1500, the executable program 410 operating on, or in communication with, smart television 118 enables the videoconferencing to commence on the display 408 of the smart television 118.

In step 1508 of method 1500, the system application program 306 operating on, or in communication with, application server 108 sends a notification to the smart television 118 that the videoconference session has concluded.

In step 1510 of method 1500, the executable program 410 operating on, or in communication with, smart television 118 displays the original user interface 406 on the smart television 118.

Referring now to FIG. 16, steps for a patient entertainment module of some embodiments is shown and described in accordance with method 1600. In step 1602 of method 1600, the executable program 410 operating on, or in communication with, smart television 118 displays the entertainment data 226 on the user interface 408 located on the display 408 of the smart television 118.

In step 1604 of method 1600, the system application program 306 operating on, or in communication with, application server 108 accesses system entertainment data 320 stored in the database server 110 per the patient's selection.

In step 1606 of method 1600, the system application program 306 operating on, or in communication with, application server 108 sends system entertainment data 226, stored in the system database 110, to the smart television 118.

In step 1608 of method 1600, the system application program 306 operating on, or in communication with, application server 108 utilized the streaming server 114 to allow for the seamless viewing of video entertainment data 226 on the user interface 408 located on the display 408 of the smart television 118.

It will be understood that although new data in the institution's EMR system 104 and elsewhere is updated across the relevant components of the system 100 databases as such new data is received, the typical process for displaying the updated information is to refresh such data on the specific screens of the user interface 406 that the patient is accessing.

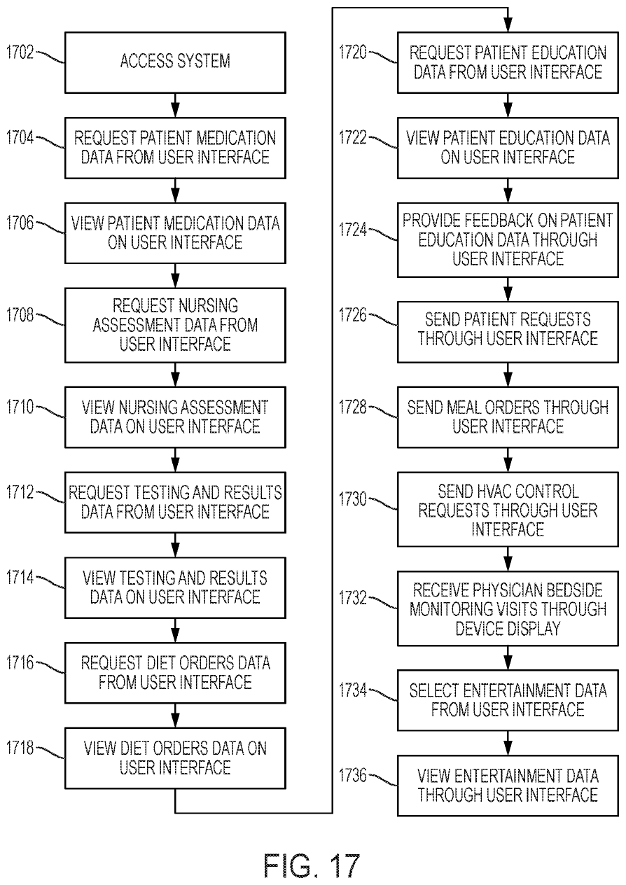

Referring now to FIG. 17, an embodiment of an interactive sharing of patient-related information method 1700 is shown. It will be understood that, in this embodiment, the method is described from the perspective of the user of the system 100.

In step 1702 of method 1700, the user accesses the system 100 through a smart television 118 located in a patient room.

In step 1704 of method 1700, the user requests patient medication data 204 from a menu item located on the user interface 406 displayed on the smart television 118.

In step 1706 of method 1700, the user views the requested patient medication data 204 on the display 408 of the smart television 118.

In step 1708 of method 1700, the user requests nursing assessment data 206 from a menu item located on the user interface 406 displayed on the smart television 118.

In step 1710 of method 1700, the user views the requested nursing assessment data 206 on the display 408 of the smart television 118.

In step 1712 of method 1700, the user requests orders and results data 208 from a menu item located on the user interface 406 displayed on the smart television 118.

In step 1714 of method 1700, the user views the requested orders and results data 208 on the display 408 of the smart television 118.

In step 1716 of method 1700, the user requests diet orders data 210 from a menu item located on the user interface 406 displayed on the smart television 118.

In step 1718 of method 1700, the user views the requested diet orders data 210 on the display 408 of the smart television 118.

In step 1720 of method 1700, the user requests patient education data 212 from a menu item located on the user interface 406 displayed on the smart television 118.

In step 1722 of method 1700, the user views the requested patient education data 212 on the display 408 of the smart television 118.

In step 1724 of method 1700, the user provides feedback relating to the requested patient education data 212 through user input menu items located on the user interface 406 on the display 408 of the smart television 118.

In step 1726 of method 1700, the user sends requests and feedback data 214 through selection of a menu item located on the user interface 406 displayed on the smart television 118.

In step 1728 of method 1700, the user sends meal orders 216 through selection of a menu item located on the user interface 406 displayed on the smart television 118.

In step 1730 of method 1700, the user sends HVAC control requests 218 through selection of a menu item located on the user interface 406 displayed on the smart television 118.

In step 1732 of method 1700, the user receives physician bedside monitoring visits through the display 408 of the smart television 118.

In step 1734 of method 1700, the user selects entertainment data 226 through selection of a menu item located on the user interface 406 displayed on the smart television 118.

In step 1736 of method 1700, the user views the requested entertainment data 226 on the display 408 of the smart television 118.

It will be understood that not all of the steps in the methods described herein are required, or must be performed in the order as described herein.

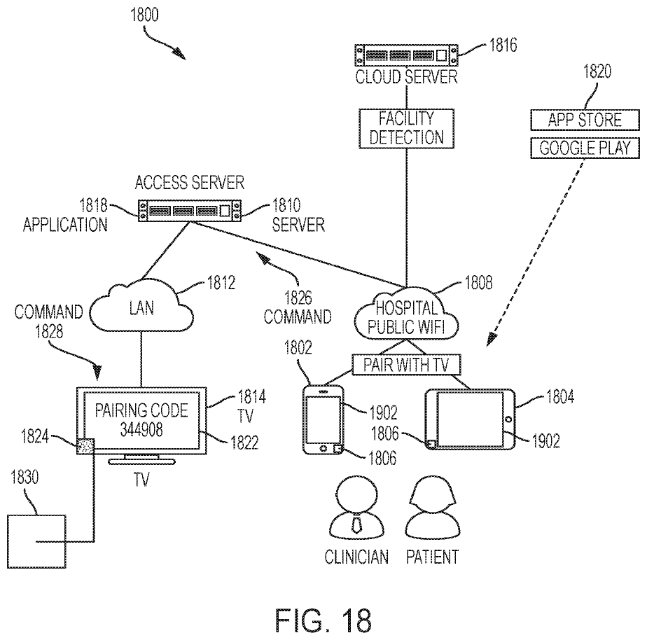

Referring now to FIG. 18, an embodiment of a command-and-control system 1800 for the control of a media and communications system is shown.

The command-and-control system 1800 includes one or more types of user devices such as a mobile phone or tablet that performs any of the functions of a computer, typically having a touchscreen interface designed to be controlled through haptic contact engagement and an operating system capable of running downloaded applications, here shown as a smartphone device 1802 and a tablet device 1804.

The command-and-control system 1800 also includes other components including a wireless local area networking system 1808, a facility application control server 1810, a facility local area network 1812, a user display terminal such as a flat-screen television 1814 or other endpoint device, a mobile application server 1820, and a cloud server 1816. It will be understood that the system 1800 may include other components as necessary for the proper functioning of the system 1800.

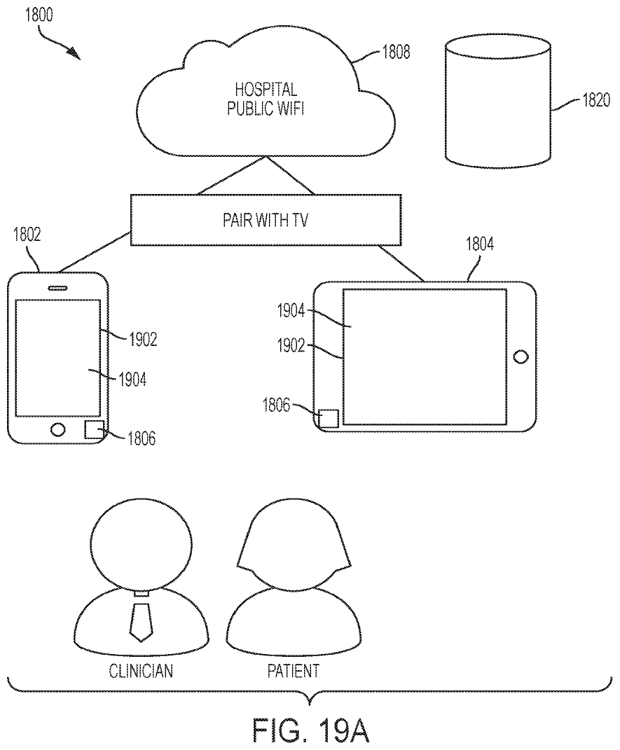

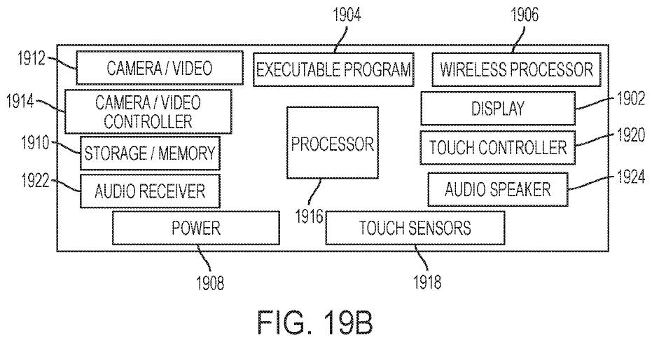

Referring now to FIG. 19A, another aspect of the command-and-control system 1800 is shown. Here, the command-and-control system 1800 utilizes user devices 1802 and 1804, which are configured to allow end users (for example, clinicians, patients, and patient family members) to interact with and otherwise utilize the command-and-control system 1800. It will be understood that any number of other devices, in addition to smartphone device 1802 and tablet device 1804 can be utilized by the system, and that smartphone device 1802 and tablet device 1804 are being offered as examples herein.

In this embodiment, each device utilizes an executable mobile application program 1806, which is stored on each device 1802 and 1804. The mobile application program 1806 is downloaded onto each device 1802 and 1804 from a mobile application server 1820 (cloud-based or local), and each device 1802 and 1804 is periodically updated through real time Internet communications between the mobile application server 1820 and each device such as smartphone device 1802 and tablet device 1804.

The mobile application program 1806 includes one or more executable program files containing instructions to perform various operations allowing the user devices to properly interact with the system as shown and described herein. For example, when operating on each user device such as smartphone device 1802 and tablet device 1804, the mobile application program 1806 causes each user device to provide a user interface 1902 to each user, and, through the hardware and software configurations of such user devices 1802 and 1804, allow each user to control each user device through haptic contact engagement on the screens 1904 of each such device.

The mobile application program 1806 is also configured to communicate with the facility application control server 1810 over a wireless local area networking system 1808 such as WiFi (in accordance with standard 802.11 of the Institute of Electrical & Electronics Engineers (IEEE), as amended from time to time) to send and receive control commands 1826 and information, and to pair with the television 1814 or other end point device that is being controlled by the user.

The facility application program 1818 operating on, or in communication with, facility application server includes one or more executable program files containing instructions to perform various operations to operate the facility application. The facility application 1818 can be any number of applications including the systems and methods described in U.S. Provisional Application No. 62/420,945, or other facility, home or business media and communications systems.

The facility application program 1818 is configured to receive control commands 1826 and information from the mobile application programs 1806 operating on, or in communication with, user devices, such as smartphone device 1802 and tablet device 1804, and send commands 1828 directly to the endpoint device 1814.

The endpoint device 1814 has an endpoint device application program 1824 that includes one or more executable program files containing instructions to perform various operations to control the endpoint device 1814 and communicate with the facility application program 1818. The endpoint device application program 1824 is configured to receive commands 1828 from the facility application 1818 and control the content and navigation of what appears on the endpoint device 1814 display 1822. It will be understood that the endpoint device application program 1824 can be stored on an endpoint device control box 1830, or in another storage and control device or location, rather than on components located on or within the endpoint device 1814 itself.

It will be further understood that each of the command-and-control system 1800 components contains non-transient computer readable storage mediums, or other storage mediums, on which such components are capable of storing information including executable and non-executable computer code, related source code, course code, binary files, application program interfaces (APIs), and/or other executable code or instructions. It will be further understood that communication between any or all of the command-and-control system 1800 components can be performed through operation of one or more application program interfaces (APIs) located on or accessible through any of the system 1800 components, including in conjunction with any application programs stored or otherwise utilized by the system 1800.

Referring now to FIG. 19B, further details of the components of a smartphone device 1802 or tablet device 1804 are shown. In this embodiment, smartphone device 1802 and/or tablet device 1804 includes a number of components including, but not limited to, a display 1902, an executable program 1904, processor 1906, power supply (battery or hardwire) 1908, storage memory component 1910, camera (with two-way video and audio capability) 1912, and camera/video controller 1914, wireless processor 1916, touch sensors 1918, touch controller 1920, audio receiver 1922, and audio speaker 1924. It will be understood that these are examples of components utilized with the command-and-control system 1800, but it will be further understood that the smartphone 1802 or tablet 1804 devices can include any number of other components that can be utilized by the command-and-control system 1800 in accordance with the manners described herein and otherwise.

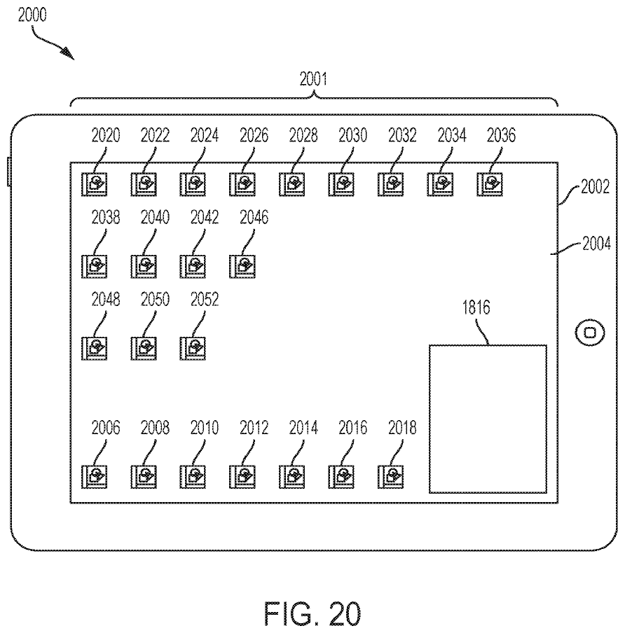

Referring now to FIG. 20, further aspects of devices 1802 and 1804 are shown, here using tablet device 1804 to illustrate various control features. Tablet device 1802 has a display 2002, and the mobile application program 1806 operating on tablet device 1804 contains executable instructions to display a graphical user interface 2004 on the display 2002. The graphics and command icons displayed on the display 2002 can vary in terms of visual features and organizations, depending on how the mobile application program 1806 is programmed. Such display can be in menu format (with drop down options), icon format (with icons arranged in various ways on the display of the smartphone device 1802), or a combination of both.

In this embodiment, the graphical user interface 2004 provides various control icons 2001 visually displayed as icons on the interface 2004. Such control icons 2001 are provided to control the navigation among, and content of, what is being displayed on a television 1814 or other end point device screen 1822. Such control icons 2001 include, but are not limited to, an on/off control 2006 configured to control turning the endpoint device application program 1824 and/or endpoint device 1814 on or off; a search control 2008 configured to control the search function/magnifying glass where the user is allowed to type in words or letters and navigate more quickly within a particular screen or page having certain content, such as within an education library, movie category, or medication list shown on the endpoint device 1814 display 1822; a keyboard control 2010 configured to provide letters, numbers, page up/down, caps lock/shift, backspace/delete, enter, calculator, allowing the user to enter and send data to the facility application server 1810 through the facility application program 1818; a download control 2012 configured to provide a button (such as an arrow) that when clicked on (through haptic engagement) allows a user to download that particular information asset (such as a handout, pdf, video, or other information) from the application server 1810 or other server within the facility system; a mousepad control 2014 configured to allow haptic touch-application manipulation of the cursor on the screen, like a mouse would on a computer; and a swipe left/right control 2016 configured to allow the user to swipe to move from application to application being displayed, for example, on the endpoint device 1814, or quickly advance/scroll right/left through a number of information assets in a collection such as movies, education data, or other items.

Such control icons 2001 also include a scrolling up/down control 2018 configured to allow the user to quickly advance/scroll up/down through a number of information assets in a collection such as education titles or dietary items; a page up/down control 2020 configured to allow the user to quickly advance/scroll up/down (as above) through pages of information assets; a channel up/down control 2022 configured to allow the user to navigate the controls of the television 1814 or other end point device; an up/down, right/left and select arrows control 2024 configured to allow the user to incrementally move the focus of navigation and control 1 space up/down/left/right and choose it with the “select” button; an information button control 2026 configured to provide a button/icon such as a circled “I” that, when clicked on, would expand with more information, allowing the access and viewing of information on medications, dietary, movies, education, and other items, including program descriptions displayed on the smartphone device 1802 interface 2004 while viewing the content on the television 1814 or other end point device; and an information icon control 2028 which is configured to resemble the information button control 2026 but which is displayed on the television 1814 or other end point device screen 1822 (discretely at the bottom/side of the screen 1822) allowing, as with information button control 2026, the access and viewing of information on medications, dietary, movies, education, and other items, including program descriptions while viewing the content on the television 1814 or other end point device.

Such control icons 2001 also include a movie viewing control 2030 configured to provide commands/trick modes (e.g., Fast Forward, Rewind, Play/Pause, Stop, Save); a closed captioning control 2032 configured to provide several closed captioning options, toggling or turning on/off with the same button; a guide control 2034 configured to allow the user to go directly to the Electronic Programming Guide and display information about the television shows; a number keys control 2036 configured to provide channels, amounts (like in food ordering), and related items; a pointer control 2038 configured to allow the user to directly access certain area in the application or TV (banners, Information button, and related items; and a quick buttons control 2040 configured to provide users with access to popular features such as movies, dining, or home page.

Such control icons 2001 also include an exit control 2042 configured to allow the user to exit the application, movie, or other like application or content; a mute button control configured to allow the user to mute sound; a last control 2044 configured to allow the user to go directly to the last channel or could display list or graphics along bottom of screen showing “trail” of last viewed channels or applications; a favorites control 2046 configured to allow the user to manually access favorite channels/apps as entered by user or determined by most views; a home button control 2048 that allows the user to navigate to the home page of the respective application; an internet button control 2050 configured to allow the user to navigate to Internet (URL of choice); a settings control 2052 configured to allow the user to change device inputs, customize look of the application; and a data entry control 2054 configured to present a data entry box and keyboard to allow the user to put input notes or access a website.

It will be understood that the icons on the user interface 2004 can include all or a portion of the controls shown on the display of the endpoint device 1814. Thus, what is shown on the user interface 2004 is highly customizable based on the needs and desires of the institution or organization utilizing the system 1800. It will be understood that what is in fact displayed to the user on the user interface 2004, particularly in terms of control icons, is determined by the device application programs 1806 operating on each user device, in coordination with the facility application program 1818.

It will be further understood that the system 1800 is also configured to allow a user to navigate with his or her mobile device of choice (e.g., a smartphone device 1802, tablet device 1804, wearable, or other related items) in conjunction with, if desired, the traditional remote/pillow speaker, based on the needs of the user and/or institution. Thus, navigation could simultaneously be controlled with one paired device, e.g. smartphone device 1802, along with multiple unpaired devices, such as a pillow speaker or wireless remote.

It will be further understood that the system 1800 could also be configured to connect with Adaptive Devices such as sip/puff controls (that is, when a user sips on a straw or exhales into a tube as a way to provide commands into the system 1800); modularize certain components through a single button that brings up trick modes; and provide a “simplified” version with less features/functions to those who need less complex navigation, like Adaptive Devices such as sip/puff. For example, users with certain neurological handicaps (e.g. stroke or Parkinson's disease) may also use assistive technology to actuate the system.

It will be further understood that the system 1800 could also be configured to provide a Customer Relationship Management (CRM) component that would help clients use, manage and analyze (in a hospital setting) patient data throughout their relationship (inpatient and outpatient). An example use case is the ability to push previously stored patient preferences (e.g. entertainment, education, adaptive UI, language, “favorites”-either by frequency of user behavior or pre-defined by user) and adjust the user interface (UI). Another use case is recording and sending/storing patient device information (Operating System, phone number, email, credentials and related items) to hospital for follow-up/post discharge contact. It will be understood that collecting the above data can be a function of the endpoint device application, the mobile application program 1806, or integrated combination of both.

It will be further understood that the system 1800 could also be configured to store and report on user activity (button clicks, quick buttons and related items) for continuous quality improvement of the application; run multiple applications in parallel (such as email, text, and the Internet), running on personal devices having button/icon for easy access; and have the ability to have more than one device control the television 1814 or other end point device or facility application program 1818, including the ability to allow a family member of a user (such as a patient in a healthcare facility) to navigate with his or her own device should the originally paired device lose battery charge or otherwise become inoperative because of malfunction, choice, or otherwise.

It will be further understood that the system 1800 could also be configured to allow users to personalize the user interface of each device in color or layout styles; “wipe”/purge data when leaving the institution (such as in a hospital/health care system environment); allow for voice commands and/or voice recognition to navigate and turn on/off, such as through a microphone button; provide the ability to be controlled in multiple languages, including voice recognition in user's chosen language; and allow for user devices to display a prompt to sync their device with the television 1814 or other end point device when within a predefined distance from the television 1814 or other end point device.

It will be understood that the above-references features and controls are by way of example only, and the user interface can include other features and controls. It will be further understood that the pillow speaker controls, other remote controls, or controls including voice control or sip/puff can perform such functions or other functions in parallel with the systems and methods described herein.

It will be further understood that the mobile application program 1806 can be configured to display any number of other controls 2006 to control or otherwise interact with the facility application program 1818 operating on, or in communication with, facility application server 1810. It will be further understood that the mobile application program 1806 is configured to send and receive information and media content, such as text messages, video content, audio content, real-time communications, and other content.

The facility application program 1818 operating on, or in communication with, facility application server 1810 is configured to receive commands from the mobile application program 1806, analyze the information, and send instruction to the appropriate end-point device (in this case a flat screen television 1814 or other end point device) for the room and bed associated with a patient. The application 1824 running on the end-point device 1814 is configured to receive the information from the facility application program 1818 and perform the navigation desired, display the appropriate screen/feature, store/modify information stored for that patient (e.g. add personal notes for themselves), or perform the desired function.

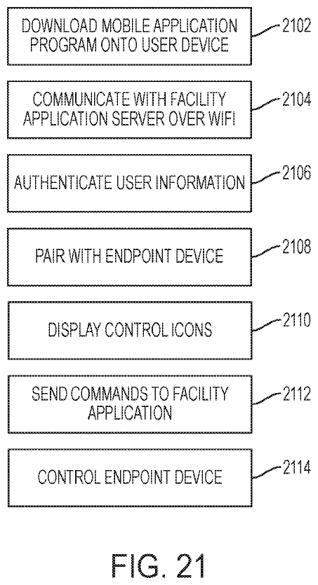

Referring now to FIG. 21, a method 2100 for controlling a media and communications system is shown.

In step 2102 of method 2100, a user downloads onto a smartphone device 1802 a mobile application program 1806 from a mobile application server 1820 over the Internet and/or a wireless local area networking system 1808.

In step 2104 of method 2100, the mobile application program 1806 operating on, or in communication with, smartphone device 1802 communicates over a secured wireless local area networking system 1808 with the facility application server 1810. Such secured protocols may include WEP, WPA, WPA2 or other security that the organization may have implemented.

In step 2106 of method 2100, the user information is authenticated. For example, in a hospital setting, the information provided in this step could be the name of the patient along with visit/account number or medical record number.

In step 2108 of method 2100, the smartphone device 1802 is paired with the television 1814 or other end point device. For example, in a hospital setting, the information provided in this step is the room number. This step 2104 could be done via a QR scan on the TV in the patient room.

In step 2110 of method 2100, once authentication is complete, the appropriate control icons 2001 are displayed on the interface 2014, as determined by the mobile application program 1806 and facility application program 1818 on what controls will populate the display 2004. At this stage the user is able to start controlling the television 1814 or other end point device via the control icons 2001 or otherwise.

In step 2112 of method 2100, commands from the mobile application program 1806 are sent to the facility application program 1818 via APIs. These commands can be activated as a result various functions, including those described herein, such as through haptic contact engagement with the command icons 2001 through 2052, sip/puff, voice control, and/or other activations.

In step 2114 of method 2100, the command information received by the facility application program 1818 is processed by the facility application program 1818 and sent to the television 1814 or other end point device for appropriate action via APIs, resulting in user control over the television 1814 or other end point device

In step 2116 of method 2100, if the information received from the mobile application program 1806 is not directly related to display on the television 1814 or other end point device, the facility application program 1818 might directly act on the data submitted to appropriately store/modify information stored for that patient (e.g. add personal notes for themselves) or trigger the activity desired by the specific command via other sets of APIs or interfaces (e.g. change room temperature).

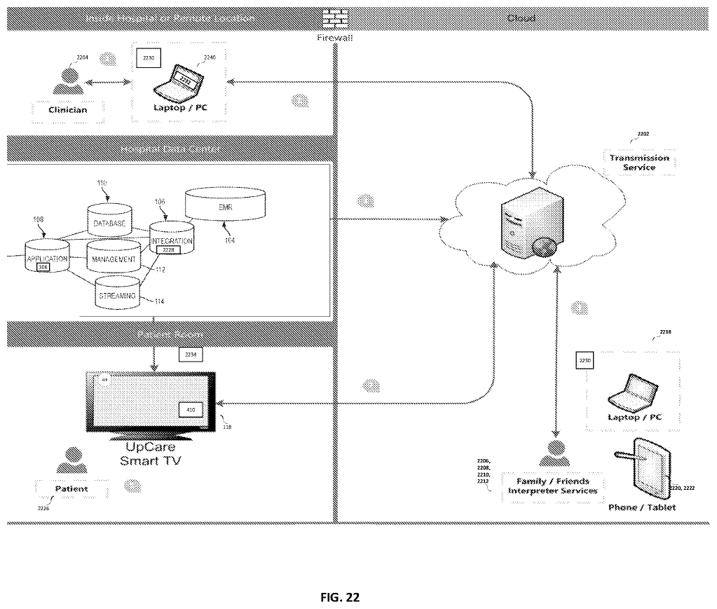

Referring now to FIG. 22 another aspect of the system 100 is shown. Here, the system 100 includes a transmission platform 2202, an electronic medical records database (EMR) 104, an integration server 106, an application server 108, a database server 110, a management server 112, and a streaming server 114. In an embodiment, the transmission platform 2202 is configured to communicate with the infrastructure of a given healthcare institution. It is understood that in an embodiment the transmission platform 2202 provides and receives video and/or audio content between users. Examples of transmission platforms 2202 include, but are not limited to, the following commercial transmission platforms: Zoom, Pexip, Caregility, Vidyo and other transmission platforms.

It is further understood that in an embodiment the transmission platform 2202 utilized by the system 100 is configured to be compliant with The Health Insurance Portability and Accountability Act of 1996 and amendments thereto (collectively “HIPAA”). The transmission platform 2202 is responsible for independently ensuring its platform is in-line with the HIPAA Security Standards.

In an embodiment, the transmission platform 2202 is configured to communicate with end user devices including devices utilized by a clinician 2204, a family member of a patient 2206, a friend of a patient 2208, an interpreter service 2210, or other third-parties 2212 seeking to conduct a video conference with a patient 2226 located within a healthcare institution. It is understood that the end user devices 2204, 2206, 2208, 2210, and 2212 might be laptops 2218 and 2240 smart phones 2220, smart televisions 118, smart tablets 2222, or other intelligent devices that are configured to store an executable program 2230 of, or otherwise utilize and communicate with, a subject transmission platform 2202. In an embodiment, the transmission platform 2202 contains operational instructions to access or receive information transacted with various end user devices (e.g., 2204, 2206, 2208, 2210, and 2212). Such information includes, but is not limited to, information relating to a patient 2226 that an end user seeks to initiate and conduct a video conference with. For example, such information might include the patient's 2226 medical record number, visit/account number, name, room number, the bed number, the clinician's 2204 name and role, and/or other information.

In the case of a clinician 2204 initiating a call to a patient 2226, in an embodiment, the clinician 2204 utilizes a clinician device 2240, which is integrated with or is otherwise communicatively coupled to the institution's hardware and software infrastructure, including as described herein, which includes, among other things, the institution's electronic medical records database (EMR) 104. In an embodiment, the EMR 104 contains information relating to, for example, the patient's medical record number, visit/account number, name, room number, bed number, and/or other information. In an embodiment, the clinician's 2204 call to a patient 2226 utilizes a transmission platform 2202 and the system 100 as described herein.

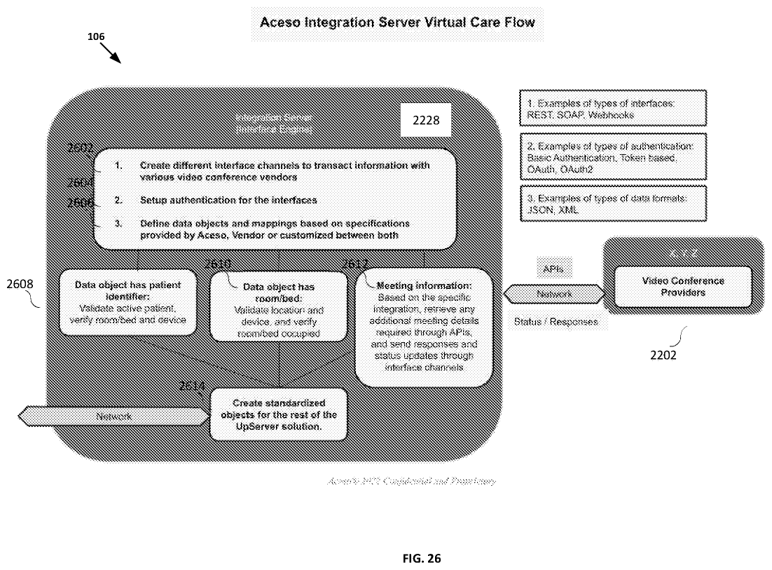

Referring now to FIG. 26, further description of the integration server 106 is shown. In an embodiment, an integration server 106 located, for example, within the system 100 infrastructure, is configured to communicate with the transmission platform 2202. In an embodiment, the integration server 106 is a virtual machine server configured to utilize one or more storage memory components 222 and utilize, interact with, or otherwise reside within the infrastructure of each given healthcare institution. It will be understood that other servers that are not virtual machine servers could be utilized for integrating with a transmission platform 2202. For example, the subject “integration” server could be a standard server known in the art that is located within the healthcare institution or at another location. The virtual and other server arrangements described herein are provided as examples, but the arrangement of servers could be made in a multitude of other ways, including through one or more additional virtual machine servers or other standard servers with storage memory components operating within a cloud-based server network, and/or other server arrangements that would allow the system 100 to operate in the manners described herein.

In an embodiment, a modular integration algorithm 2228 operating on, or in communication with, integration server 106 and related components contains operational instructions to access and parse information transacted with the transmission platform 2202. Such information includes, but is not limited to, information relating to the patient 2226 that is the subject of a given video conference. For example, such information might include the patient's medical record number, visit/account number, the name, room number, bed number, and/or other information.

In an embodiment, a modular integration algorithm 2228 operating on, or in communication with, integration server 106 (or another server or other such device) and related components is configured to create 2602 different interface channels for receiving data from a transmission platform 2202. For example, the different interface channels might include a TCP/IP Channel (e.g., HL7 channel), a Web Service channel (e.g., REST channel or SOAP channel), or a Web Hooks channel, SMTP (e.g. email), File System IO, FTP/SFTP.

In an embodiment, the modular integration algorithm 2228 is configured to set up 2604 authentication mechanisms for multiple interface channels. The authentication mechanisms can include password-based authentication, multi-factor authentication, basic authentication, certificate-based authentication, token-based authentication, OAuth, OAuth2, and other authentication mechanisms. In an embodiment, the modular integration algorithm 2228 is further configured to define 2606 data objects and mappings based on specifications as defined in a particular implementation including as set forth by the institution or transmission platform 2202. The data objects can be defined according to different data formats including JavaScript Object Notation (JSON), Extensible Markup Language (XML), HL7, delimited file formats, or other formats.

The defined data objects are comprised of multiple data points based on the information received from the transmission platform 2202. It will be understood that different transmission platforms 2202 transmit different data points. For example, the defined data objects might include data points corresponding to a medical record number, visit/account number, patient name, a patient room number, patient bed number, meeting ID and other meeting credentials, and other corresponding data points.

The modular integration algorithm 2228 is configured to validate the data object depending on the particular data points comprising the data object. For example, if the data object contains a patient 2226 identifier (e.g., medical record number, visit/account number, patient name), the modular integration algorithm 2228 is configured to validate 2608 the patient identifier belongs to an active patient 2226 and further verify the corresponding patient's 2226 room number, bed number, and device. The patient 2226 identifier and the patient's 2226 room number, bed number, and device are validated against data received from the EMR 104 and stored in the database 110. In an embodiment, if the data object has a data point reflecting a patient's 2226 room number and bed number, the modular integration algorithm 2228 is configured to validate 2610 the location and the device corresponding to the patient's 2226 room number bed number and further verify the identified room number and bed number are occupied.

In an embodiment, the modular integration algorithm 2228 is configured to use an Application Programming Interface (API) to retrieve any additional meeting information required but not part of the initial request (e.g. passcode, room) or to send meeting status updates and responses to the transmission platform 2202. For example, the modular integration algorithm 2228 is configured to receive and send information 2612 (e.g. patient declined call) based on the integration with the specific transmission platform 2202. The modular integration algorithm 2228 is further configured to standardize 2614 the defined data objects for utilization in the system 100. This approach allows us to rapidly create custom integrations with new transmission platforms 2204 while not having to change the main application program.

The formats for each of these functions vary by transmission platform 2202 (for example, the formats for Pexip, Zoom, and Caregility each differ from each other for these functions in one or more ways). The modular integration algorithm 2228 translates these formats, and parses and integrates the accompanying data allowing the transmission platform 2202 to be utilized by the smart television 118, one or more user devices 2218, 2220, 2222, and the overall system 100. The user devices might comprise a laptop/PC 2218, a phone 2220 or a tablet 2222. In an embodiment, the modular integration algorithm 2228 and integration server 106 are configured to receive and identify data from the different transmission platform 2202 providers on a single server (which is particularly beneficial to an institution that might have multiple video conferencing providers).

In an embodiment, an integration server 106 and modular integration algorithm 2228 are also configured to send the transmission platform 2202 any unique tokens created for a video call along with various statuses during the call.

In an embodiment, the system also includes an application server 108 as described herein. In an embodiment, the application server 108 and system application program 306 operating on, or in communication with, application server 108 are configured to receive data from an integration server 106 after the data from the transmission platform 2202 is parsed and integrated as described herein. In an embodiment, once the application server 108 receives the data, it and the system application program 306 are configured to check and determine whether the device (e.g., smart television 118) is online based on activity from the device, and if the device is offline, a message is sent to the integration server 106 so it can create the appropriate error response. If the device is online, the application server 108 and system application program 306 are configured to send the data received from the integration server 106 to the correct smart television 118 or other device. The availability of the smart television 118 can be based on the activity of the smart television 118 in a given time interval determined by the healthcare system or another party. For example, in one embodiment the time interval used to measure the smart television's availability might be set to thirty minutes. In this embodiment, the system application program 306 operating on, or in communication with, application server 108 will check to see if the smart television 118 has been active in the past thirty minutes.

The application server 108 and system application program 306 are also configured to create a clinician portal 2232. This can be useful if the transmission platform 2202 does not provide one or is otherwise not configured to identify a patient name, room, device or other information that may be stored in the subject EMR 104. In such a case, in an embodiment, the portal 2232 would be configured to retrieve and present the patient and room/bed information and also create the video meeting using the transmission platform's 2202 APIs. That information can then be routed directly through the application server 108 or through the integration server 106 depending on use case and workflow.

In an embodiment, the smart television 118 has an executable program 410 operating on or in communication with the smart television 118. In an embodiment, the executable program 410 operates on the smart television 118 or a Smart Set-Back-Box 2234 attached to the a conventional television.

In an embodiment, the smart television 118 and executable program 410 are configured to render the user interface code/portals that patients (or others at the bedside) interact with. In an embodiment, this code listens for any notifications and data from the system 100 (including from the integration server 106 and/or application server 108) relating to the transmission platform 2202 that are meant for the smart television 118. Once the executable program 410 receives the data, it is able to use that data to determine the identity of the subject transmission platform 2202 and type of connection that needs to be made. Based on the provider or the specific solution at a given health system, this could be using a protocol like WebRTC on the smart television's 118 browser or it could be via direct connection to a specific camera device. The executable program 410 can then communicate directly with the transmission platform's 2202 APIs or through the application server 108 and integration server 106 for various meeting features.

The actual audio/video feeds, any PTZ (pan, tilt, zoom) controls, and in some cases interface data points happen directly between the Smart TV 118 and the transmission platform 2202. Once the video connection is made, in the case of protocols like WebRTC, the executable program 410 transacts data relating to the video connection directly with the transmission platform 2202 using APIs. In the case of a connection to a camera device via an HDMI port, the executable program 410 communicates with the transmission platform's 2202 APIs through the application server 108 and integration server 106. Specifically, the executable program 410 transacts audio/video data directly with the transmission platform 2202 or with the Smart TV 118. Additionally, any signals from controls related to audio/video data are sent directly from the transmission platform 2202 to the executable program 410 or the Smart TV 118. For example, if a clinician 2204 chooses to pan, tilt, or zoom the camera in the patient 2226 room or mute their microphone, the corresponding signal related to that selection is transmitted from the transmission platform 2202 to the executable program 410 or Smart TV 118.

In an embodiment, the executable program 410 includes a layer of code that allows the visual presentation and display of the data from the transmission platform 2202 to be customizable to meet the needs and look-and-feel of the health system and support the various use-cases and workflows. For example, in an embodiment, such use-cases and workflows can include providing a notification of a video call request to the patient, options for the patient to accept or decline the call, the ability for a clinician to directly start a video call if they need to monitor a patient, and other controls based on the solution.

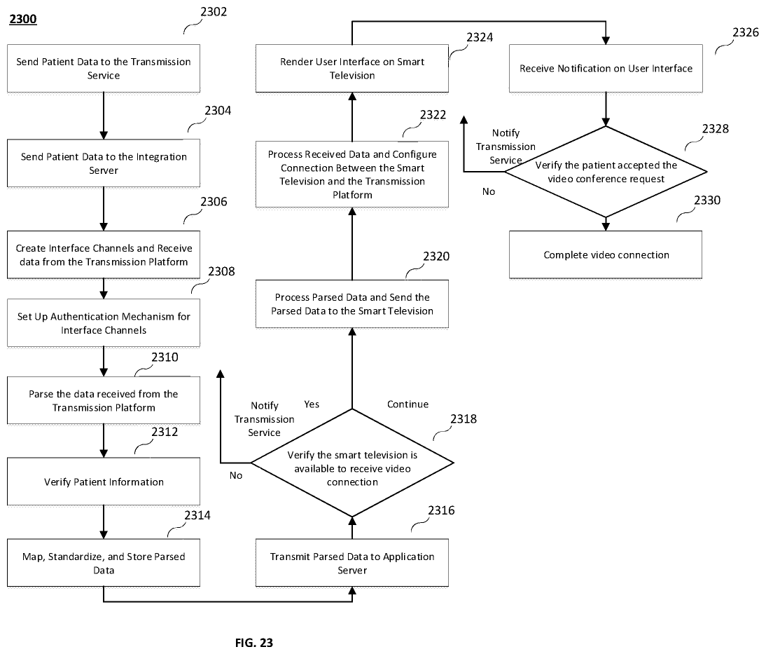

Referring now to FIG. 23, a method 2300 for facilitating a video conference between a clinician 2204 and a patient 2226 is shown and described.

In step 2302 of method 2300, a clinician 2204 seeking to initiate a video conference with a patient 2226 sends information identifying the patient 2226 to the transmission platform 2202 (e.g. Pexip, Zoom, Caregility). It will be understood the information might be the patient's 2226 medical record number, visit/account number, name, or room and bed number.

In an embodiment, the transmission platform 2202 does not provide its own portal. In such embodiments, the system application program 306 operating on, or in communication with, application server 108 is configured to create a portal 2232 allowing the clinician 2204 to identify the patient 2206 for video conferencing. The portal 2232 created by the system application program 306 operating on, or in communication with, application server 108 is configured to retrieve and present patient and room/bed information using information stored in the database 110 received from the EMR 104.

In step 2304 of method 2300, the transmission platform 2202 formats the data received by the clinician 2204 in a format specified by the system 100 and sends the data to the modular integration algorithm 2228 operating on, or in communication with, integration server 106.

In step 2306 of method 2300, the modular integration algorithm 2228 creates different interface channels for receiving information from the transmission platform 2202. The different channels might include a TCP/IP channel, a Web Service channel (e.g., REST or SOAP channel), or a Webhooks channel. Once configured, the interface channels listen for communication from the transmission platform 2202.

In step 2308 of method 2300, the modular integration algorithm 2228 sets up authentication for the different interface channels. It will understood that different transmission platforms 2202 might require different authentication mechanisms. For example, the different transmission platforms 2202 might use various authentication mechanisms such as password-based authentication, multi-factor authentication, certificate-based authentication, token-based authentication, or another authentication method. It will be understood that the authentication will be performed with respect to the headers of the data packets received from the transmission platform 2202.

In step 2310 of method 2300, the modular integration algorithm 2228 parses the data received from the transmission platform. It will be understood that step 2310 occurs only when the authentication performed in step 2308 is successful. The parsing performed by the modular integration algorithm 2228 includes defining data objects. It will be understood that the data objects can be defined according to different data formats (e.g., XML or JSON).

The defined data objects contain multiple data points corresponding to the information received from the transmission platform 2202. It will be understood that different transmission platforms 2202 transmit different data points. Examples of the data points provided by the transmission platform 2202 might include a medical record number, visit/account number, patient name, a patient room number, patient bed number, meeting credentials, and other corresponding data.

To illustrate, in embodiments where the transmission platform 2202 is Zoom, the initial setup consists of registering within the Zoom environment for a given healthcare system. A channel created during step 2306 defined on the integration server 106 using Webhooks The initial setup also involves creating Zoom room locations within the Zoom environment for that healthcare system so the clinicians 2204 can use the Zoom portal 2232 to send an invite to a specific zoom room device, after they look up the patient's 2226 location in the EMR 104. When the clinician 2204 initiates a video conference in step 2302, a meeting invite gets sent to the integration server 106 over the secure connection. For authentication, Zoom requires the channel to be reachable via a public domain defined for the integration server 106 over a secure https transaction (i.e. requiring an SSL certificate on the integration server). For example, the authentication in step 2308 is performed using OAuth and a token. The defined data objects created in step 2310 consists of data points such as the environment's account-id, operator/requestor, participant, meeting-id, meeting description, and a uuid (unique identifier for transactions between the integration server and Zoom). The interface then retrieves the zoom room location and in some cases additional information like a passcode using APIs. Notifications reflecting whether the patient accepted, rejected or missed a meeting invite are sent from the integration server 106 to Zoom.

In step 2312 of method 2300, the modular integration algorithm 2228 verifies the patient 2226 information using the parsed data. For example, in a hospital setting, the information used to verify the patient 2226 in this step could be the room and bed of the patient along with visit/account number or medical record number.

It will be understood that the verification performed by the modular integration algorithm 2228 varies depending on the data points comprising the data objects defined in step 2310. For example, if the data object contains a patient 2226 identifier (e.g., medical record number, visit/account number, patient name), the modular integration algorithm 2228 is configured to validate the patient identifier belongs to an active patient 2226 and further verify the corresponding patient 2226 room number, bed number, and device. If the data object has a data point reflecting a patient's 2226 room number and bed number, the modular integration algorithm 2228 is configured to validate the location and the device corresponding to the patient's 2226 room number and bed number and further verify the identified room number and bed number are occupied.

In step 2314 of method 2300, the modular integration algorithm 2228 parses data, obtains any missing information and maps it to standardize the data that is utilized by the system 100. To illustrate, the various transmission platforms 2204 might send meeting details using different formats. For example, one transmission platform 2204 might send the meeting details as part of a URL that needs to be parsed in order to create the standardized object; another transmission platform 2204 might send meeting details using separate fields for the meeting ID and password; and another transmission platform 2204 might send meeting details that require using another API to get the passcode.

Similarly, various transmission platforms 2204 send location information using different formats. Some transmission platforms 2204 send the patient Acct #or MRN, requiring that the database be queried to determine the facility, room and bed; other transmission platforms 2204 might send a location defined in the transmission service's 2204 system through an API using a meeting-id or uuid from the invite, requiring the system 100 to then look at the mapping for the room and bed (which corresponds to the EMR 104).

Additionally, some transmission platforms 2204 require that a unique reference number be generated to use for future transactions. For transmission platforms 2204 that do not require generating such an identifier, the system 100 will still generate an identifier and store it in the database 110.

In step 2316 of method 2300, the modular integration algorithm 2228 transmits the parsed data to the system application program 306 operating on, or in communication with, application server 108.

In step 2318 of method 2300, the system application program 306 operating on, or in communication with, application server 108 verifies whether the smart television 118 identified in the parsed data for a given patient 2226 is available to receive a video connection. Verifying the availability of the smart television 118 includes checking to see if the smart television 118 is online based on whether activity from the device has occurred within a certain time interval. The time interval used to measure the smart television's 118 availability is a configurable parameter. For example, in one embodiment the time interval used to measure the smart television's availability might be set to thirty minutes. In this embodiment, the system application program 306 operating on, or in communication with, application server 108 will check to see if the smart television 118 has been active in the past thirty minutes.

If the system application program 306 operating on, or in communication with, application server 108 determines the smart television 118 is available, the method 2300 continues. If not, the system application program 306 notifies the modular integration algorithm 2228 and the method 2300 ceases until the data is updated or otherwise corrected and the method 2300 commences again beginning at step 2302.

In step 2320 of method 2300 the system application program 306 operating on, or in communication with, application server 108 processes the data received from the modular integration algorithm 2228 and sends the data to the executable program 410 operating on, or in communication with, smart television 118.

In step 2322 of method 2300, after the data sent from the modular integration algorithm 2228 is received the executable program 410 operating on or in communications with the smart television 118 processes the received data to determine which transmission platform 2202 is being used. Based on the transmission platform 2202 determined or otherwise identified, the executable program 410 will configure the appropriate connection. For example, in an embodiment the transmission platform 2202 might require the executable program 410 to establish a direct connection with a camera device (two-way video and audio capability) 428 attached to the smart television 118. In another embodiment, the transmission platform 2202 might require the executable program 410 to set up a connection using a particular protocol like WebRTC, for example.

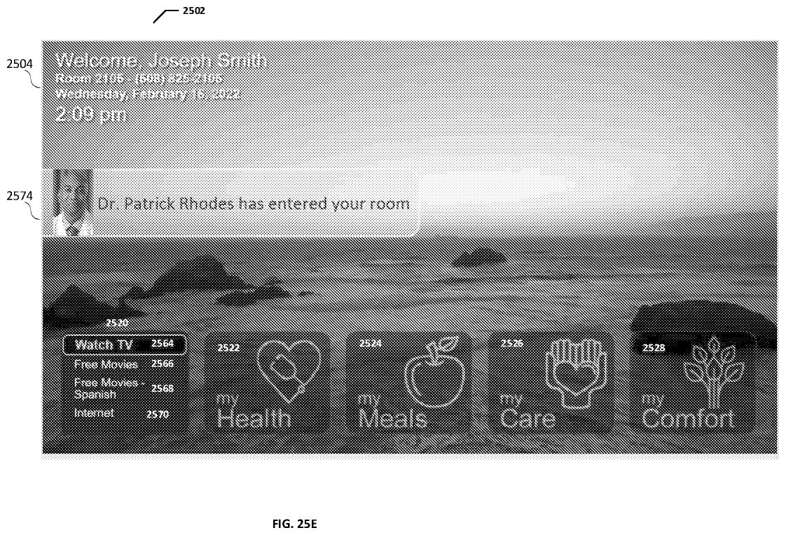

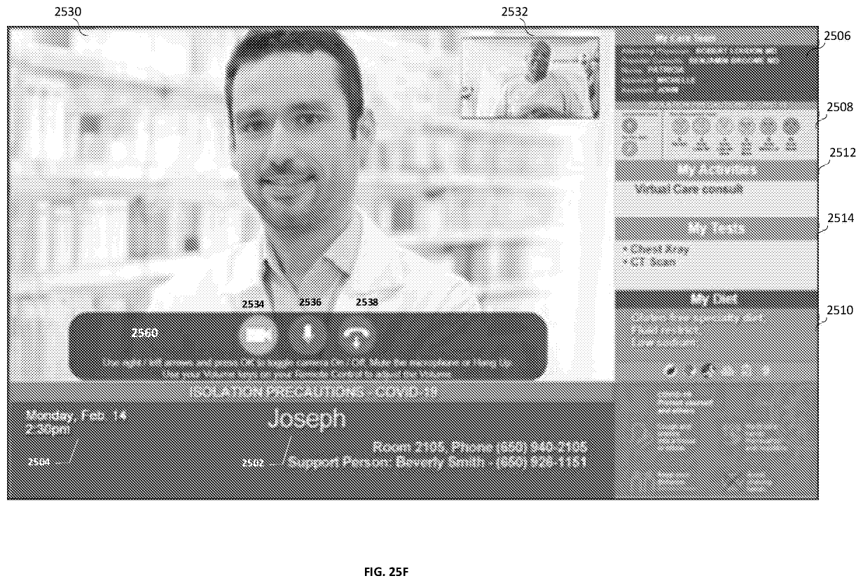

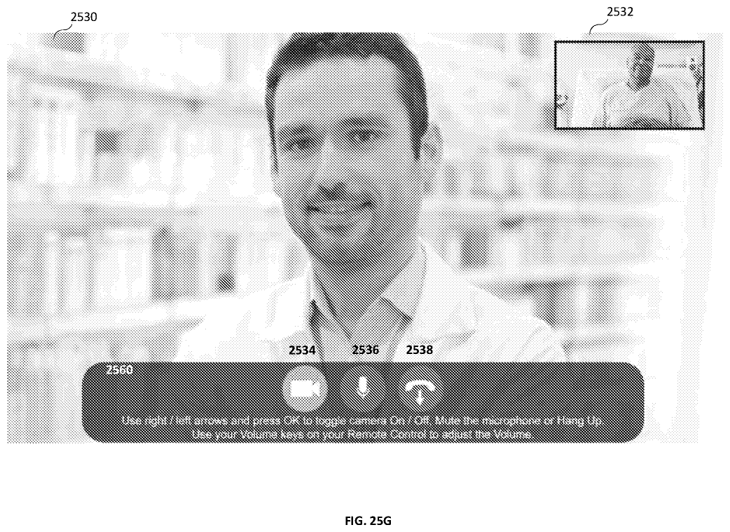

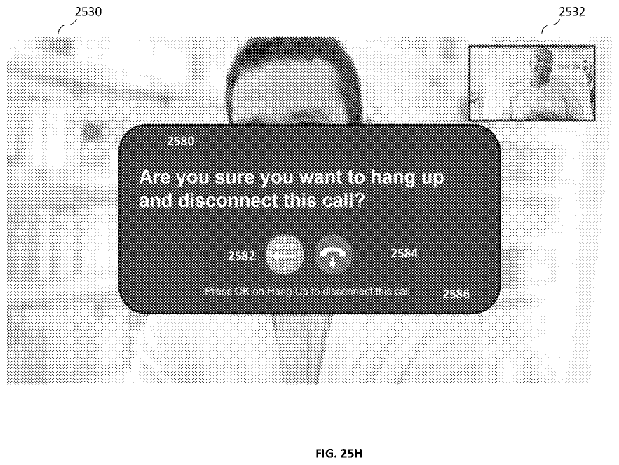

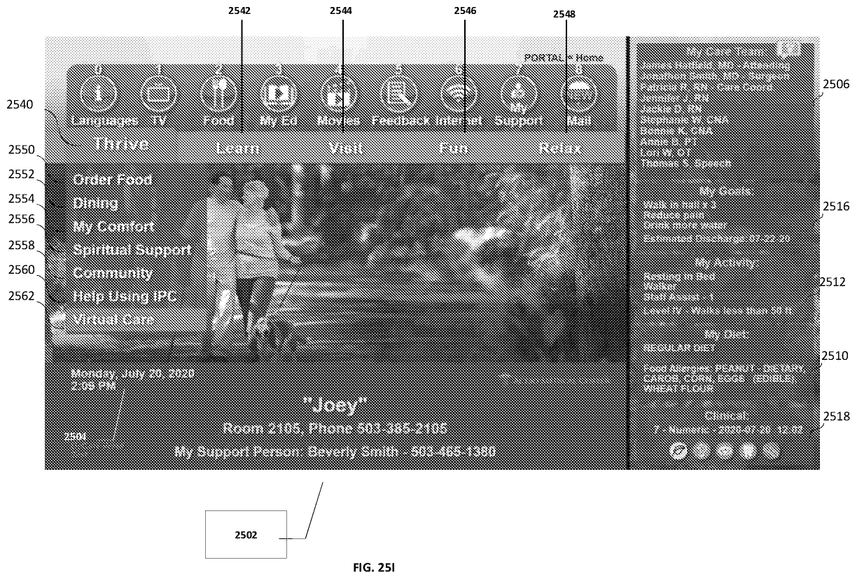

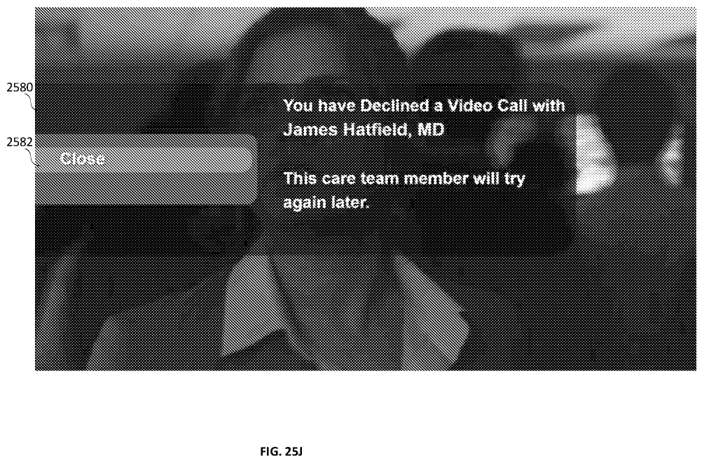

In step 2324 of method 2300 the executable program 410 operating on or in communication with the smart television 118 renders the appropriate user interface 406 on the smart television 118. The user interface might include detailed information concerning a patient's care team 585, physiology 586, diet 587, activities and schedule 588, tests 589, and other clinical information 595. See, e.g., FIGS. 25A-25J. Every hospital system can have multiple User Interfaces based on nursing unit, hospital wing, etc. The information displayed on the user interface 406 highlights the claimed invention's flexibility for accommodating different workflows along with enhanced capabilities of integration with the EMR and other 3rd party vendors. In some embodiments, the user interfaces 406 are rendered on the Smart TVs 118. This is accomplished using specific programming for running within the constraints such SoC (system on chip) devices to enable Smart TVs 118 to interact with the hardware components. This is an advantage over the user interfaces provided by transmission platforms 2202 which are designed for devices like computers, laptops, mobile devices and as such will not render on Smart TVs 118 in the patient room setting.

In step 2326 of method 2300 the modular integration algorithm 2228 operating on, or in communication with the integration server 106 sends a notification to the user interface 406 of the smart television 118 alerting the patient 2226 that a clinician 2204 is requesting a video conference. In some embodiments, the user interface 406 of the smart television 118 displays a “pop-up” screen identifying the individual initiating the call and allows to patient 2226 to either accept or reject the video conference using their remote controller 426. In some embodiments, the “pop-up” screen will be a photo of the individual initiating the call. See, e.g., FIG. 25C.

It will be understood that in some embodiments the clinician 2204 is able to initiate a video conference without providing the patient 2226 the ability to accept or decline the call on the user interface 406 of the smart television 118. When a clinician 2204 initiates a video conference in this manner, the user interface 406 of the smart television 118 provides a notification indicating that a clinician 2204 is monitoring the patient. See, e.g., FIG. 25E.

In step 2326 of method 2300, if necessary, the modular integration algorithm 2228 operating on, or in communication with, integration server 106 verifies whether the patient 2226 accepted the video conference and sends the appropriate response to the transmission platform 2202.

It will be understood that if the video conference is accepted, the modular integration algorithm 2228 operating on, or in communication with, integration server 106 will transmit any unique tokens created for the video conference to the transmission platform 2202.

In some embodiments, if the patient 2226 declines the video conference request or fails to answer, the clinician 2204 will be given a chance to send another video conference request to the patient 2226.

It will be further understood that the throughout the duration of the video conference, the modular integration algorithm 2228 operating on, or in communication with, integration server 106 can send updates to the transmission platform 2202 regarding the status of the call based on the specifications of the integration.

In step 2328 of method 2300, if the patient 2226 accepts the video conference request, the transmission platform 2202 completes the video connection with the executable program 410 operating on, or in communication with, smart television 118. It will be understood that additional components attached to the smart television 118 are configured to allow two-way audio and video communication between the patient 2226 and the clinician 2204 through its various hardware and software features as shown and described herein. After the call is initiated, the executable program 410 will communicate audio/video data directly with the transmission platform 2202 or smart television 118.

In an embodiment the patient 2226 is presented with the options to change the settings of the video call. For example, the patient 2226 might be permitted to toggle their camera 428 and/or audio on and off throughout the call using their remote controller 426. Any selections made by the patient 2226 related to the audio/video settings will cause the executable program 410 to send the appropriate signal to the transmission platform 2202. It will be appreciated that if the camera 428 is enabled, the user interface 406 of the smart television 118 will provide the patient 2226 with a message indicating the camera 428 is on.

Referring now to FIG. 25A-25J several embodiments of the user interface 406 displayed by the system application program 306 on each smart television 118 is shown.

Referring now to FIG. 25A an embodiment of a system user interface 406 is shown. Here, patient 2226 name and room information 2502 is displayed, along with the day, date and time 2504. The displayed includes detailed information concerning a patient's 2226 care team 2506, pain levels 2508, special diet and allergies 2510, activities and schedule 2512, and tests 2514. A video conference feed 2530 received from the transmission platform 2202 is displayed. Another video conference feed 2532 received from the camera 428 of the patient's device is overlaid over the video conference feed 2530 in a display window smaller than that of the display window for the video conference feed 2530. Actionable icons for toggling the camera on/off 2534, muting the microphone 2536, and ending the call 2538 are also overlaid on the video feed 2530. It will be understood that once selected, each actionable icon will cause the executable program 410 to send the appropriate signal the transmission platform 2202. For example, selecting the camera 2534 icon will cause the video feed from the patient's camera 428 to turn off or on, depending on status of the camera 428 prior to selection. Selecting the microphone 2536 icon will cause the microphone of the patient's device to be muted such that sound is not transmitted to the clinician 2204. Selecting the icon for ending the call 2538 will transmit a message to the transmission platform 2202 for disconnecting the call.

Referring now to FIG. 25B an embodiment of a system user interface 406 is shown. Here, patient 2226 name and room information 2502 is displayed, along with the day, date and time 2504. The displayed information includes detailed information concerning a patient's 2226 care team 2506, pain levels 2508, diet 2510, activities and schedule 2512, and tests 2514. A video conference feed 2530 received from the transmission platform 2202 is displayed. Another video conference feed 2532 received from the camera 428 of the patient's device is overlaid over the video conference feed 2530 in a display window smaller than that of the display window for the video conference feed 2530. Actionable icons for toggling the camera on/off 2534, muting the microphone 2536, and ending the call 2538 are overlaid on the video feed 2532. Additionally, instructions 2560 for how to select each actionable icon is displayed. The instructions 2560 explain what happens upon selecting each actionable icon. It will be understood that once selected, each actionable icon will cause the executable program 410 to send the appropriate signal the transmission platform 2202. For example, selecting the camera 2534 icon will cause the video feed from the patient's camera 428 to turn off or on, depending on status of the camera 428 prior to selection. Selecting the microphone 2536 icon will cause the microphone of the patient's device to be muted such that sound is not transmitted to the clinician. Selecting the icon for ending the call 2538 will transmit a message to the transmission platform 2202 for disconnecting the call.

Referring now to FIG. 25C an embodiment of a system user interface 406 is shown. Here, patient 2226 name and room information 2502 is displayed, along with the day, date and time 2504. The displayed information includes detailed information concerning a patient's 2226 care team 2506, diet 2510, activities and schedule 2512, clinical information 2518, and other nursing assessment data 2516. A pop-up message 2562 is displayed indicating that a clinician 2204 is requesting a video conference call. The pop-up message 2562 includes actionable options for either accepting 2564 or rejecting 2566 the incoming call. It will be understood that if the patient 2226 selects the option for accepting 2564 the incoming call, the transmission service 2202 will complete the video connection. It will be further understood that if the patient 2226 selects the option for rejecting 2566 the incoming call the video connection will not be completed.

Referring now to FIG. 25D an embodiment of a system user interface 406 is shown. Here, patient 2226 name and room information 2502 is displayed, along with the day, date and time 2504. The displayed information includes detailed information concerning a patient's 2226 care team 2506, activities and schedule 2512, clinical information 2518, and other nursing assessment data 2516. A video conference feed 2530 received from the transmission platform 2202 is displayed. Additional media 2590 is overlaid over the video conference feed 2530 in a display window smaller than that of the display window for the video conference feed 2530. The additional media 2590 might be video data provided by the clinician 2204 and received from the transmission platform 2202. Examples of additional media 2590 might include test results, x-rays, or patient education materials.

Referring now to FIG. 25E an embodiment of a system user interface 406 is shown. Here, patient 2226 name and room information 2502 is displayed, along with the day, date and time 2504. General menu items relating to enjoyment 2520, health 2522, meals 2524, care 2526, and comfort 2528 are displayed. The enjoyment menu item 2520 can have subset menu items for the patient to choose from including general television 2564, free movies 2566, free movies in foreign languages 2568, and internet 2570. A notification 2574 is displayed indicating that a patient 2226 is current being monitored by a clinician 2204.

Referring now to FIG. 25F an embodiment of a system user interface 406 is shown. Here, patient 2226 name and room information 2502 is displayed, along with the day, date and time 2504. The displayed information includes detailed information concerning a patient's care team 2506, pain levels 2508, diet 2510, activities and schedule 2512, and tests 2514. A video conference feed 2530 received from the transmission platform 2202 is displayed. Another video conference feed 2532 received from the camera 428 of the patient's device is overlaid over the video conference feed 2530 in a display window smaller than that of the display window for the video conference feed 2530. Actionable icons for toggling the camera on/off 2534, muting the microphone 2536, and ending the call 2538 are overlaid on the video feed 2530. Additionally, instructions 2560 for how to select each actionable icon are displayed. The instructions 2560 further explain what happens upon selecting each actionable icon. It will be understood that once selected, each actionable icon will cause the executable program 410 to send the appropriate signal the transmission platform 2202. For example, selecting the camera 2534 icon will cause the video feed from the patient's camera 428 to turn off or on, depending on status of the camera 428 prior to selection. Selecting the microphone 2536 icon will cause the microphone of the patient's device to be muted such that sound is not transmitted to the clinician 2204. Selecting the icon for ending the call 2538 will transmit a message to the transmission platform 2202 for disconnecting the call.

Referring now to FIG. 25G a video conference feed 2530 received from the transmission platform 2202 is displayed. Another video conference feed 2532 received from the camera 428 of the patient's device is overlaid over the video conference feed 2530 in a display window smaller than that of the display window for the video conference feed 2530. Actionable icons for toggling the camera on/off 2534, muting the microphone 2536, and ending the call 2538 are overlaid on the video feed 2530. Instructions 2560 for how to select each actionable icon are displayed. The instructions 2560 further explain what happens upon selecting each actionable icon. It will be understood that once selected, each actionable icon will cause the executable program 410 to send the appropriate signal the transmission platform 2202. For example, selecting the camera 2534 icon will cause the video feed from the patient's camera 428 to turn off or on, depending on status of the camera 428 prior to selection. Selecting the microphone 2536 icon will cause the microphone of the patient's device to be muted such that sound is not transmitted to the clinician 2204. Selecting the icon for ending the call 2538 will transmit a message to the transmission platform 2202 for indicating the patient wishes to disconnect the call.

Referring now to FIG. 25H an embodiment of a system user interface 406 is shown. A video conference feed 2530 received from the transmission platform 2202 is displayed. Another video conference feed 2532 received from the camera 428 of the patient's device is overlaid over the video conference feed 2530 in a display window smaller than that of the display window for the video conference feed 2530. A pop-up message for ending the video conference call 2580 is displayed. The pop-up message 2580 contains actionable icons for returning to the call 2582 or ending the call 2584. The pop-up message 2580 further includes instructions 2586 for how to select each actionable icon 2582 and 2584. It will be understood that upon selection, the actionable icon for returning to the call 2582 will cause the video conference call to continue. It will be further understood that upon selection, the actionable icon for ending the call 2584 will cause the executable program 410 to notify the transmission service 2202 to disconnect the video conference.

Referring now to FIG. 25I an embodiment of a system user interface 406 is shown. The displayed information includes detailed information concerning a patient's 2226 care team 2506, diet 2510, activities and schedule 2512, clinical information 2518, and other nursing assessment data 2516. General menu items relating to the patient services 2540, patient education 2542, patient feedback 2544, enjoyment 2546, and patient relaxation 2548 are displayed. The patient services menu item 2540 can have subset menu items for the patient 2226 to choose from including ordering food 2550, dining 2552, comfort 2554, spiritual support 2556, community 2558, help 2560, and video conferencing 2562. It will be understood that once selected, each actionable menu item will direct the patient to a new page allowing that user to further interact with the system 100 with respect to subject matter relating the menu item.

The patient education menu item 2542 can have subset menu items for the patient 2226 to choose from including the General Health Library of Education videos, education videos assigned to the patient, and educational TV channels.

The patient feedback menu item 2544 can have subset menu items for the patient 2226 to choose from including submitting feedback about their care, submitting expectations about their care, and information on the pharmacy.

The patient enjoyment menu item 2546 can have subset menu items for the patient 2226 to choose from including watching TV, free movies, music, and internet.

The patient relaxation menu item 2548 can have subset menu items for the patient 2226 to choose from including scenic TV, sleep enhancement, pet therapy, and request for relaxation services.

Referring now to FIG. 25J an embodiment of a system user interface 406 is shown. A message 2580 is displayed. The message 2582 indicates that the patient declined a video conference call from clinician. An actionable selection allowing the user to close 2580 the message 2582 is displayed.

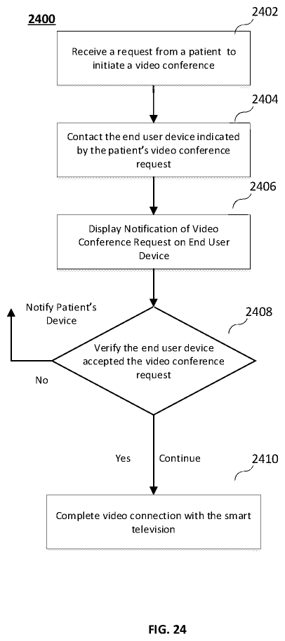

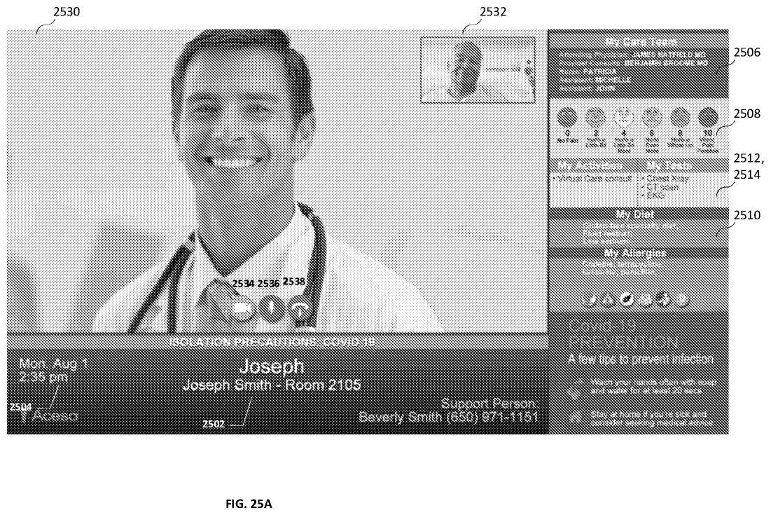

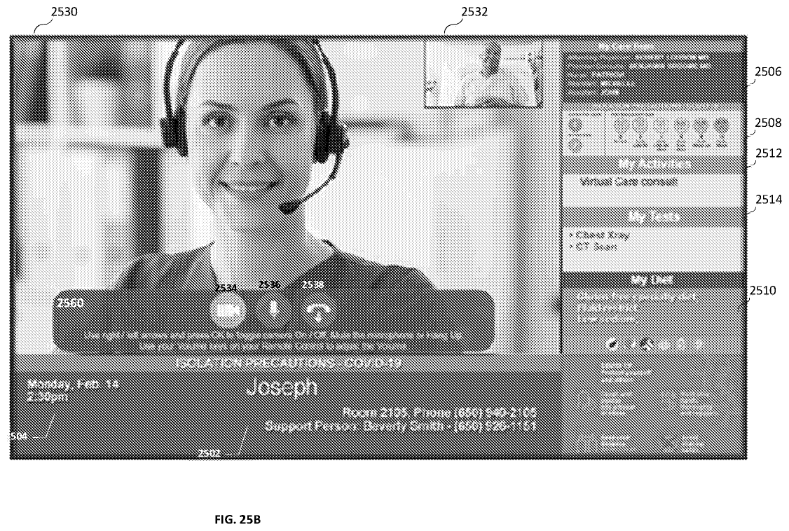

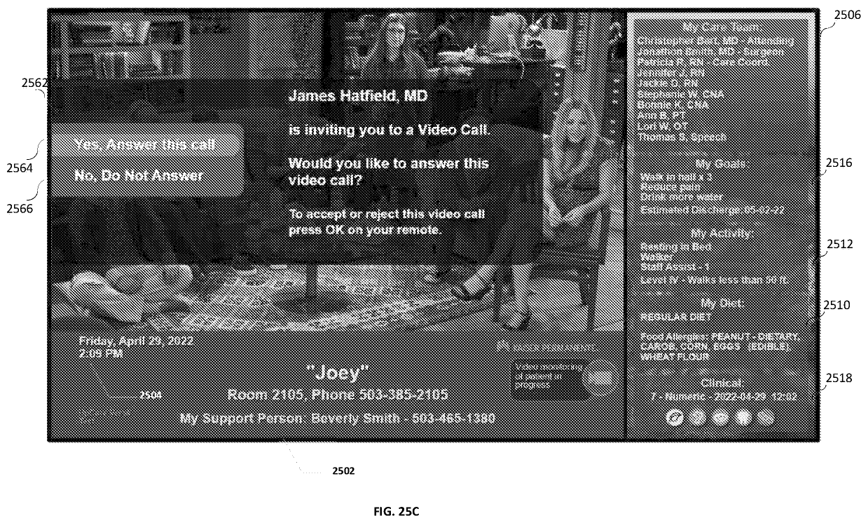

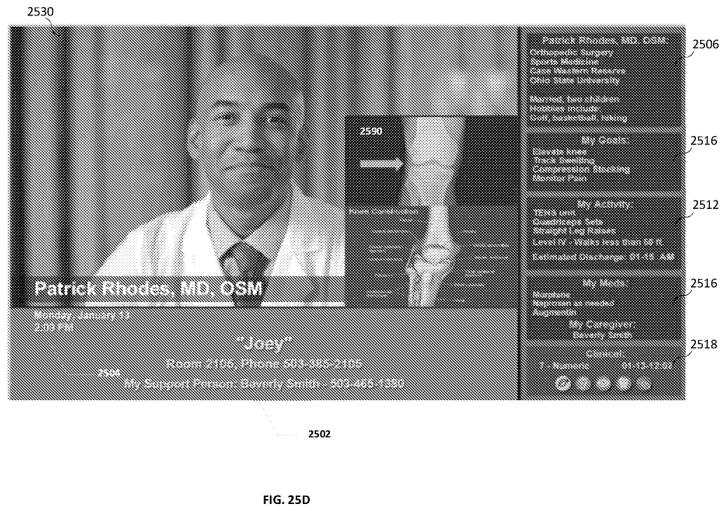

Referring now to FIG. 24, a method 2400 for facilitating a video conference between a patient device 118 and an end user device is shown and described. It will be understood that end user device might be a device belonging to a family member of a patient 2206, a friend of the patient 2208, an interpreter service 2210, or other third-party 2212.

In step 2402 of method 2400, a patient 2226 initiates a video conference call from the system 100 that utilizes a certain transmission platform 2202. In an embodiment, the patient 2226 initiates a video conference call using the executable program 410 operating on, or in communication with, smart television 118. The video conference request might include data identifying the family member or an interpreter service the patient wishes to initiate a video conference with. For example, the transmitted data might include a mobile number, email address, or direct web address corresponding to an end user (e.g., 2206, 2208, 2210, and 2212). In some embodiments, the patient 2226 initiates the video conference call by sending the transmitted data to through the application server 108 to the integration server 106 which forwards the data to the transmission platform 2204 or the end user (e.g., 2206, 2208, 2210, and 2212).

In an embodiment, depending on transmission platform 2204, the transmission platform 2204 might be actively waiting for a notification or request from the end user (e.g., 2206, 2208, 2210, and 2212) to initiate a video call with the patient 2226.

Depending on the given transmission platform 2202, the system 100 might be required to use an authentication mechanism. For example, the transmission platform 2202 might use an authentication mechanism such as password-based authentication, multi-factor authentication, certificate-based authentication, token-based authentication, or another authentication method.

In step 2404 of method 2400, the transmission platform 2202 contacts the end user device (e.g., 2206, 2208, 2210, and 2212) according to the information included in the patient's video conference request and transmits a notification to the user interface 406 of the end user device (e.g., 2206, 2208, 2210, and 2212) indicating that the patient 2226 requested a video conference.

In step 2406 of method 2400 the user interface 406 of the end user device (e.g., 2206, 2208, 2210, and 2212) displays a notification indicating that the patient 2226 requested a video conference. The notification displayed on the user interface 406 provides the option to accept or decline the video conference request.

In step 2408 of method 2400 the transmission platform 2202 verifies whether the end user (e.g., 2206, 2208, 2210, and 2212) accepted the video conference request. In some embodiments, if the end user (e.g., 2206, 2208, 2210, and 2212) declines the video conference request or fails to answer, the patient 2226 will be given a chance to send another video conference request to the end user (e.g., 2206, 2208, 2210, and 2212).

In step 2410 of method 2400, if the video conference request is accepted, the transmission platform 2202 completes the video connection with the executable program 410 operating on or in communication with the smart television 118. In some embodiments, the smart television 118 sends the video connection information through the application server 109 to the integration server 106.

It will be understood that additional components attached to the smart television 118 are configured to allow two-way audio and video communication between the patient 2226 and other end users (e.g., 2206, 2208, 2210, and 2212) through its various hardware and software features as shown and described herein. In some embodiments the patient is presented with the options to change the settings of the video call. For example, the patient 2226 might be permitted to toggle their camera 428 and/or audio on and off throughout the call using their remote controller 428. Any selections made by the patient 2226 related to the audio/video settings will cause the executable program 410 to send the appropriate signal to the transmission platform 2202. It will be appreciated that if the camera 428 is enabled, the user interface 406 of the smart television 118 will provide the patient 2226 with a message indicating the camera 428 is on.

It will be understood that not all of the steps in the methods described herein are required, or must be performed in the order as described herein.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A communication system for facilitating video conferencing comprising:
one or more servers, wherein the one or more servers are communicatively coupled to a transmission platform for facilitating communications with one or more in-room devices, wherein the transmission platform is configured to send and receive video-related data; and
one or more processors containing instructions which, when executed by the one or more processors, cause the one or more processors to:
create communication interface channels for receiving patient-related data from the transmission platform,
receive, through the communication interface channel, a message containing patient-related data from the transmission platform,
authenticate the message,
parse the patient-related data contained in the authenticated message, wherein the parsing includes converting the patient-related data into one or more data objects having a format compatible with the system,
transmit the standardized patient-related data to one or more in-room devices, and
configure a connection between the in-room device and the transmission platform.

2. The communication system of claim 1 wherein the one or more processors are further caused to verify the patient-related data using an Electronic Medical Records (EMR) system.

3. The communication system of claim 1 wherein the communication interface channel is one or more of a TCP/IP channel, a web service channel, a REST channel, a SOAP channel, or web hooks channel.

4. The communication system of claim 1 wherein the parsing the patient data further comprises retrieving video-related data from the transmission platform using an application programming interface.

5. The communication system of claim 1 wherein the data object comprises one or more of a patient identifier, a room number, or a bed location.

6. The communication system of claim 1 wherein the patient-related data is authenticated using one or more of password-based authentication, multi-factor authentication, certificate-based authentication, or token-based authentication.

7. A method for facilitating video conferencing comprising:
creating communication interface channels for receiving patient-related data from the transmission platform,
receiving, through the communication interface channel, a message containing patient-related data from the transmission platform,
authenticating the message,
parsing the patient-related data contained in the authenticated message, wherein the parsing includes converting the patient-related data into one or more data objects having a format compatible with the system,
transmitting the standardized patient-related data to one or more in-room devices, and
configuring a connection between the in-room device and the transmission platform.

8. The method of claim 7, further comprising verifying the patient-related data using an Electronic Medical Records (EMR) system.

9. The method of claim 7 wherein the communication interface channel is one or more of a TCP/IP channel, a web service channel, a REST channel, a SOAP channel, or web hooks channel.

10. The method of claim 7 wherein the parsing the patient data further comprises:
retrieving video-related data from the transmission platform using an application programming interface.

11. The method of claim 7 wherein the data object comprises one or more of a patient identifier, a room number, or a bed location.

12. The method of claim 7 wherein the patient-related data is authenticated using one or more of password-based authentication, multi-factor authentication, certificate-based authentication, or token-based authentication.

13. A method for facilitating video conferencing comprising:
receiving end-user-related data from an in-room device,
creating communication interface channels for transmitting end-user-related data to a transmission platform,
communicating video-related data with one or more in-room devices,
configuring a connection between the in-room device and the transmission platform.

14. The method of claim 13, further comprising verifying the patient-related data using an Electronic Medical Records (EMR) system.

15. The method of claim 13 wherein the communication interface channel is one or more of a TCP/IP channel, a web service channel, a REST channel, a SOAP channel, or web hooks channel.

16. A method for normalizing patient-related data comprising:
creating communication interface channels for receiving patient-related data from a transmission platform,
receiving, from the communication interface channel, a message containing patient-related data from the transmission platform,
authenticating the message, and
parsing the patient-related data contained in the authenticated message, wherein the parsing includes converting the patient-related data into one or more data objects having a format compatible with the system.

17. The method of claim 16 wherein the communication interface channel is one or more of a TCP/IP channel, a web service channel, a REST channel, a SOAP channel, or web hooks channel.

18. The method of claim 16 wherein the parsing the patient data further comprises retrieving video-related data from the transmission platform using an application programming interface.

19. The method of claim 16 wherein the data object comprises one or more of a patient identifier, a room number, a bed location.

20. The method of claim 16 wherein the patient-related data is authenticated using one or more of password-based authentication, multi-factor authentication, certificate-based authentication, or token-based authentication.
